SEQUENCE LISTING (4 sequences)

SEQ ID NO: 1 - protein, 200 aa, Oryza sativa
MDRDESLGTQPLTGRRVRADTRHPVYRGIRLRSGKWVSEIREPGKSSRIWLGTYPTPEMAAAAYDAAALALRGADAALNFPGTATSRPAPASGSPDDIRAAAAAAAAMIGSGHRGNQRAADASTSRAATAAPEAAVAAGAGDQKRVVDEDDVFEMPRLLVSMAEGLMMSPPRLSPSTDGVGGVSPEDDEDEDGMSLWNHS

SEQ ID NO: 2 - DNA, 603 bp, Oryza sativa
atggatcgagacgagagcttgggcacgcagccattgaccgggaggcgggtgcgcgcggacacaaggcacccggtgtaccgcggcatccggctccgcagcggcaagtgggtctcggagatccgcgagccgggcaagtccagcaggatctggctcggcacgtacccgacgcccgagatggccgccgcggcgtacgacgccgcggcattggcgctgcgcggcgccgacgccgcgctcaacttccccggcacggccacgtcgcgcccggcgccagcctccggctcccccgacgacatccgcgcagcggccgcggccgccgccgcgatgatcggctccggtcaccgtggcaaccagcgcgccgccgacgcgagcacgtctcgcgcggcgacggcggcgccggaggccgccgtcgccgctggggcgggcgatcagaagcgcgtcgtggacgaggacgacgtcttcgagatgccgcggctgctggtgagcatggccgagggcttgatgatgagcccgccgaggctgagcccctcgacggacggcgtcggcggcgtgtcgccggaggacgacgaggacgaggacggcatgagcctgtggaaccattcctga

SEQ ID NO: 3 - protein, 199 aa, Oryza sativa
MDRDESLGTQPLTGRRVRADTRHPVYRGIRLRSGKWVSEIREPGKSSRIWLGTYPTPEMAAAAYDAAALALRGADAALNFPGTATSRPAPASGSPDDIRAAAAAAAAMIGSGHRGNQRAADASTSRAAPAPEVAVAAGAGDQKRVVDEDDVFEMPRLLVSMAEGLMMNPPRLSPSTDGVGGVSPEDDEDEDGMSLWNHS

SEQ ID NO: 4 - protein, 257 aa, Sorghum bicolor
MAVAVAVVSGTGIAHLHFLHVLLPEIPGFSGEEQRGSVPVCPECPGQMEREQEQEAGTAQQLLGRRVRADTRHPVYRGIRYRGGKWVSEIREPRKSNRIWLGTYPAPEMAAAAYDAAALALRGAEAALNFPGAAMSRPAPASCSPDDIRAAAAAAAAAVIGRSHSPQVGGEAAAGGGCGASTWSSGAGAQGQVPEHRAGDRRIVDEDDVFQVPRLLAGMAEGLMMSPPRLVGPATDGAVLLEEDGSEDGVVSLWDHS

(19) United States
(12) Patent Application Publication
(10) Pub. No.: US 2014/0223604 A1
(43) Pub. Date: Aug. 7, 2014

(54) CROP PLANTS WITH IMPROVED WATER USE EFFICIENCY AND GRAIN YIELD AND METHODS OF MAKING THEM

(71) Applicants: Andy Pereira, Fayetteville, AR (US); Madana M.R. Ambavarm, Norwood, MA (US); Utlwang Batlang, Gaborone (BW)

(72) Inventors: Andy Pereira, Fayetteville, AR (US); Madana M.R. Ambavarm, Norwood, MA (US); Utlwang Batlang, Gaborone (BW)

(21) Appl. No.: 14/117,422

(22) PCT Filed: May 14, 2012
(86) PCT No.: PCT/US12/37730

§ 371 Date: Nov. 13, 2013

Related U.S. Application Data
(60) Provisional application No. 61/485,683, filed on May 13, 2011.

(51) Int. Cl. C12N 15/82

(52) U.S. Cl. CPC C12N 15/8273; C12N 15/8261
USPC 800/290; 800/298; 435/320.1; 800/278

(57) ABSTRACT

The present invention provides crop plants and methods for improving grain yield of crop plants both under environmental stress as well as optimal conditions, as well as methods of making such plants. In particular, the present invention provides methods that result in crop plant stability under multiple environments by increasing biomass, water use efficiency and abiotic stress tolerance. Embodiments provide a transgenic crop plant comprising a chimeric gene that comprises: a transcription regulatory sequence active in plant cells and a nucleic acid sequence encoding a HYR protein, wherein such HYR protein comprises the sequence of SEQ ID NO:1, a sequence with at least 70% similarity to SEQ ID NO:1, a sequence encoding an ortholog protein, a sequence encoding a homologous protein, a functional fragment, and any combination thereof.

### CROSS-REFERENCE TO RELATED APPLICATIONS

This application is a National Stage application under 35 USC §371 of Application No. PCT/US12/37730, filed May 14, 2012, which application claims priority to and the benefit of the filing date of U.S. Provisional Application No. 61/485,683, filed on May 13, 2011, the disclosures of which are hereby incorporated by reference herein in their entireties.

### REFERENCE TO SEQUENCE LISTING SUBMITTED IN COMPUTER READABLE FORM

The present application contains a Sequence Listing which has been submitted in ASCII format by way of EFS-Web and is hereby incorporated by reference herein in its entirety. The ASCII file was created Feb. 4, 2014 and named VTIP58sequence020414.txt, which is 7.94 kilobytes in size and which is identical to the substitute paper copy filed Mar. 17, 2014 for this national stage application. The submission of the Sequence Listing in this national stage application does not include matter which goes beyond the disclosure of International Application No. PCT/US12/37730 as filed.

### BACKGROUND OF THE INVENTION

1. Field of the Invention

The present invention relates to the field of transgenic plants. More specifically, embodiments of the invention provide crop plants and methods for improving grain yield of crop plants both under environmental stress as well as optimal conditions, as well as methods of making such plants. In particular, the present invention provides methods that result in crop plant stability under multiple environments by increasing biomass, water use efficiency and abiotic stress tolerance.

2. Description of Related Art

The yield of crop plants is central to the well being of humans and is directly affected by the growth of plants under physical environment. Abiotic environmental stresses, such as drought stress, salinity stress, heat stress, and cold stress, are major limiting factors of plant growth and productivity. Crop losses and crop yield losses of major crops such as soybean, rice, maize (corn), cotton, and wheat caused by these stresses represent a significant economic and political factor and contribute to food shortages in many underdeveloped countries.

Plant biomass is the total yield for forage crops like rice, alfalfa, silage corn and hay. Many proxies for yield have been used in grain crops. Chief amongst these are estimates of plant size. Plant size can be measured in many ways depending on species and developmental stage, but include total plant dry weight, above-ground dry weight, above-ground fresh weight, leaf area, stem volume, plant height, rosette diameter, leaf length, root length, root mass, tiller number and leaf number. Many species maintain a conservative ratio between the size of different parts of the plant at a given developmental stage. These allometric relationships are used to extrapolate from one of these measures of size to another. Plant size at an early developmental stage will typically correlate with plant size later in development. A larger plant with a greater leaf area can typically absorb more light and carbon dioxide than a smaller plant and therefore will likely gain a greater weight during the same period. This is in addition to the potential continuation of the micro-environmental or genetic advantage that the plant had to achieve the larger size initially. There is a strong genetic component to plant size and growth rate, and so for a range of diverse genotypes plant size under one environmental condition is likely to correlate with size under another. In this way a standard environment is used as a proxy for the diverse and dynamic environments encountered at different locations and times by crops in the field.

Harvest index, the ratio of seed yield to above-ground dry weight, is relatively stable under many environmental conditions and so a robust correlation between plant size and grain yield can often be obtained. These processes are intrinsically linked because the majority of grain biomass is dependent on current or stored photosynthetic productivity by the leaves and stem of the plant. Therefore, selecting for plant size, even at early stages of development, has been used as an indicator for future potential. When testing for the impact of genetic differences on stress tolerance, the ability to standardize soil properties, temperature, water and nutrient availability and light intensity is an intrinsic advantage of greenhouse or plant growth chamber environments compared to the field. However, artificial limitations on yield due to poor pollination due to the absence of wind or insects, or insufficient space for mature root or canopy growth, can restrict the use of these controlled environments for testing yield differences. Therefore, measurements of plant size in early development, under standardized conditions in a growth chamber or greenhouse, are standard practices to provide indication of potential genetic yield advantages.

During the life cycle, plants are typically exposed to conditions of reduced environmental water content. Most plants have evolved strategies to protect themselves against these conditions of desiccation. However, if the severity and duration of the drought conditions are too great, the effects on development, growth, plant size, and yield of most crop plants are profound. Continuous exposure to drought conditions causes major alterations in the plant metabolism which ultimately lead to cell death and consequently yield losses.

Developing stress-tolerant plants is therefore a strategy that has the potential to solve or mediate at least some of these problems. However, traditional plant breeding strategies to develop new lines of plants that exhibit resistance and/or tolerance to these types of stresses are relatively slow and require specific resistant lines for crossing with the desired line. Limited germplasm resources for stress tolerance and incompatibility in crosses between distantly related plant species represent significant problems encountered in conventional breeding. Additionally, the cellular processes leading to drought, cold, and salt tolerance in model drought-, cold- and/or salt-tolerant plants are complex in nature and involve multiple mechanisms of cellular adaptation and numerous metabolic pathways. This multi-component nature of stress tolerance has made breeding for tolerance largely unsuccessful, but has also limited the ability to genetically engineer stress tolerant plants using biotechnological methods.

Drought stress is among the most serious challenges to production worldwide for the major cereals: maize (Zea mays), rice (Oryza sativa), and wheat (Triticum aestivum) (Pellergrineschi et al., 2004). Major research efforts are directed at understanding the mechanism of plant responses to drought stress in order to identify gene products that confer adaptation to water deficit. Molecular mechanisms of water stress response have been investigated primarily in the model plant species Arabidopsis thaliana (Liu et al., 1998). Upon exposure to drought stress conditions, many stress-related genes are induced, and their products are thought to function as cellular protectors from stress-induced damage (Oh et al., 2009; Shinozaki et al., 2003). The expression of stress-related genes is largely regulated by transcription factors (TF). The rice and Arabidopsis thaliana genomes code for more that 1500 TF, and about 45% of them are reported from plant-specific families (Kikuchi et al., 2003). Various drought stress studies have identified TF families with putative functions in drought including MYB, bZIP, Zinc finger, NAM, and APETALA2 (AP2) (Oh et al., 2009; Zhou et al., 2007). The AP2 family is one of the plant-specific TF whose members share a highly conserved DNA-binding domain known as AP2 (Weigel, 1995). Members of this family have been associated with various developmental processes (Chuck et al., 1998) and with stress tolerance (Haake et al., 2002; Liu et al., 1998).

Many approaches might be taken to boost intrinsic yield in plants, including increasing photosynthetic capacity, modifying plant architecture, and enhancing the rate of growth. An algorithm to identify key points of regulation for enhancing plants' rate of photosynthesis has been described (Zhu et al., 2007). The prospects for controlling photosynthetic capacity have been reviewed by Horton et al. (2000) and Long et al. (2006), and some of the specific opportunities identified are excellent targets for TF-based genetic manipulation. In addition to yield improvement, significant commercial efforts are being focused on stabilizing yield in the face of environmental pressures such as drought. Two trait areas, yield and drought tolerance, offer high-value returns on products and are major targets for TF-based genetic improvement (Century et al., 2008).

The AP2 TF CBF4 (also known as DREB1 [dehydration-responsive element-binding protein]) is probably the most studied in drought. Overexpression of CBF4 was found to lead to drought adaptation in Arabidopsis (Haake et al., 2002) and wheat (Pellegrineschi et al., 2004). Another AP2 Arabidopsis TF called HARDY was recently reported to provide enhanced drought tolerance in Arabidopsis and rice (Karaba et al., 2007). Ectopic expression of these genes confers drought tolerance and/or adaptation by modifying cellular structures of leaves and roots, CO₂ exchange, and parameters such as water use efficiency (WUE), which correlate with the transformed plants' ability to withstand drought. Taken together, these and other findings indicate that AP2 TF offers the potential to engineer plants in a way that makes them more productive under stress conditions.

Although drought stress can alter the growth and development of a plant at any time during its life cycle, water limitations during reproductive growth stages can be especially conducive to yield losses in crops like rice (Selote and Khana-Chopra, 2004) and maize (Zinselmeier et al., 1995). Accordingly, the reproductive phases in these plants should be an important stage to study for identifying stress-responsive genes that might have a protective, or yield altering, function in drought. Advances in plant genomics, including the availability of the complete genome sequence of rice, have provided an opportunity to identify stress-related TF that control drought-related traits. To this end, a genome-wide analysis of drought stress responses was conducted and led to the identification of a candidate drought-induced AP2/ERF TF in reproductive tissues.

Despite advancements in plant engineering, there are still unmet, critical and immediate needs such as the need for plants with higher grain yield even under well-watered conditions but especially for plants with higher grain yield under stressful environmental conditions such as drought-stressed conditions. Therefore, what is needed is the identification of the genes and proteins involved in these multi-component processes leading to increased growth and/or increased stress tolerance. Elucidating the function of genes expressed in stress tolerant plants will not only advance our understanding of plant adaptation and tolerance to environmental stresses, but also may provide important information for designing new strategies for crop improvement.

### SUMMARY OF THE INVENTION

The numerous limitations inherent in plant growth under stressful environmental conditions described above provide great incentive for new plants, and methods of using such plants, to provide higher crop yield. The present invention provides a method for improving grain yield of crop plants both under environmental stress as well as optimal conditions. More specifically, the present invention provides methods that result in crop plant stability under multiple environments by increasing biomass, water use efficiency and abiotic stress tolerance.

In some embodiments, the present invention provides a transgenic crop plant comprising a chimeric gene that comprises: a transcription regulatory sequence active in plant cells and a nucleic acid sequence encoding a HYR protein, wherein such HYR protein comprises the sequence of SEQ ID NO:1, a sequence with at least 70% similarity to SEQ ID NO:1, a sequence encoding an ortholog protein, a sequence encoding a homologous protein, a functional fragment, and any combination thereof.

In another embodiment, the present invention provides a chimeric gene comprising: a tissue specific-, inducible- or developmentally-regulated promoter active in plant cells and a nucleic acid sequence encoding a HYR protein, wherein such HYR protein comprises the sequence of SEQ ID NO:1, a sequence with at least 70% similarity to SEQ ID NO:1, the sequence of SEQ ID NO:3, the sequence of SEQ ID NO:4, a sequence encoding an ortholog protein, a functional fragment thereof, and any combination thereof.

Other embodiments provide a vector comprising a chimeric gene comprising: a constitutive, inducible, developmental stage-preferred, cell type-preferred, tissue-preferred, or organ-preferred promoter active in plant cells and a nucleic acid sequence encoding a HYR protein, which comprises the sequence of SEQ ID NO:1, a sequence with at least 70% similarity to SEQ ID NO:1, the sequence of SEQ ID NO:3, the sequence of SEQ ID NO:4, a sequence encoding an ortholog protein, a functional fragment thereof, and any combination thereof.

Another embodiment provides such vectors wherein the encoding nucleic acid sequence has the sequence of SEQ ID NO:2, a sequence encoding an ortholog protein, a sequence encoding a homologous protein, a functional fragment, and any combination thereof.

Another embodiment provides the vector above wherein the nucleotide sequence is operably linked with a stress inducible promoter.

Some embodiments of the present invention provide a method of using a HYR protein described in this specification to create a transgenic crop plant having one or more phenotypes selected from the group consisting of: enhanced water use efficiency, enhanced photosynthesis, enhanced biomass, enhanced grain yield, enhanced drought tolerance, and any combination thereof. The term “enhanced” in the context of this specification is used to refer to plants of the invention with higher efficiency, tolerance, yield etc. as compared with plants not having the inventive genetic advantage.

Other embodiments provide a method of using such vectors to create a transgenic crop plant with one or more phenotypes selected from the group consisting of: enhanced water use efficiency, enhanced photosynthesis, enhanced biomass, enhanced grain yield, enhanced drought tolerance, and any combination thereof.

Yet another embodiment provides a method of using a host cell comprising: a transcription regulatory sequence active in plant cells and a nucleic acid sequence encoding a HYR protein, wherein such HYR protein comprises the sequence of SEQ ID NO:1, a sequence with at least 70% similarity to SEQ ID NO:1, a sequence encoding an ortholog protein, a sequence encoding a homologous protein, a functional fragment, and any combination thereof to create a transgenic crop plant.

Some embodiments provide a seed and/or a fruit of a plant described herein.

Another embodiment of the present invention provides a product produced by a plant described in this specification and used for a foodstuff, feedstuff, a food supplement, feed supplement, cosmetic, pharmaceutical, and any combination thereof.

The features and advantages of the present invention will be apparent to those skilled in the art. While numerous changes may be made by those skilled in the art, such changes are within the spirit of the invention.

### BRIEF DESCRIPTION OF THE DRAWINGS

These drawings illustrate certain aspects of some of the embodiments of the present invention, and should not be used to limit or define the invention.

FIG. 1 is a graphical representation of the effect of drought on HYR gene expression in different developmental stages of rice WT. Data are expressed as the mean relative transcript levels in drought compared to that of well watered (log 2 ratio). Error bars represent mean±SE (n=3). Asterisks indicate significant differential expression (t-test; *, P≦0.05; **, P≦0.01).

FIG. 2 is a picture showing the selection of putative transgenic plants for hygromycin resistance: (a) wild-type; (b) to (d) putative HYR-transgenic lines; (e) PCR-genotyping for hygromycin resistance (hpt) gene in the transgenic plants.

FIG. 3 is a graphical representation of gravimetric parameters. (a) shoot biomass, and (b) WUE in wild type (WT) and HYR-transformed rice lines under well-watered and drought-stressed conditions. WUEg was calculated using the formula in Paragraph [0094]. In (b), *indicates significant difference between HYR and WT within the watering treatments. In (a), each HYR line is different than WT for both well-watered and drought-stressed plants. Error bars are standard error of the means, n=4.

FIG. 4 is a picture showing the morphology of well-watered HYR-transgenic plants compared to the wild type (WT). (a) one week after planting; (b) two weeks after planting; (c) ten weeks after planting. (1=WT, 2=HYR-2, 3=HYR-4, 4=HYR-16).

FIG. 5 is a graph of the gas exchange analysis and leaf water status of wild type (WT) and transgenic HYR lines under well-watered and drought stressed conditions: (a) Photosynthesis (Pn); (b) transpiration (E); (c) instantaneous water use efficiency (WUEi); and (d) relative water content (RWC %). Error bars are standard error of the means, n=4.

FIG. 6 is a graphical representation of (a) Percentage of relative water loss from the leaves of WT and HYR lines during progressive drought stress treatment. Values are the mean±SE (n=6); and (b) Gas exchange measurement analysis of five HYR transgenic rice lines and WT under well watered (WW) and drought (DR) stress conditions. Photosynthetic rate was measured using LI-6400XT from three leaves in each pot and the data represent ±SE of six plants in each genotype (n=6) (t-test; *, P≦0.05; **, P≦0.01).

FIG. 7 is a picture showing the effect of progressive drought on rice WT and HYR transgenic lines at vegetative stage. (a) Plants (WT and three representative independent T3 homozygous HYR lines) grown for 6-weeks in the environmentally controlled growth chambers and the phenotype of plants at ‘day-0’ of drought stress. (b) Appearance of plants after two days of drought stress (day-2). (c) Appearance of plants at ‘day-4’ of drought stress, where WT are showing about 75% RWC and HYR lines maintained 85% RWC. (d) Phenotype of plants at ‘day-6’ of progressive drought. The WT plants reached to 65% RWC and HYR lines still maintained 80% RWC.

FIG. 8 is a graphical representation showing the analysis of soluble sugars; glucose, fructose, sucrose, and total sugars in WT and HYR lines under well watered and drought stressed conditions. Errors bars are standard error of the means, n=4.

FIG. 9 is a schematic of agronomic traits of HYR plants grown under well watered conditions. Spider plots of yield components of five independent homozygous T3 lines of HYR and WT controls grown under normal conditions in the green house. Each data point represents a percentage of the mean values (n=10), and the mean values from WT controls were set at 100% as a reference.

FIG. 10 contains pictures showing the phenotypic characterization of rice HYR lines. (a) Three-weeks grown WT and five HYR transgenic rice lines used for chlorophyll estimation. (b) Leaf blade phenotypes of WT (upper lane) and HYR, showing the bright and dark green leaf surface in HYR plants (lower lane). (c) and (d), Confocal microscope images of leaves from WT and HYR, respectively. The sections were stained with 1% toluidine blue and photographed with a light microscope (40×) under identical microscopy parameters. (e) and (f), Transmission electron micrographs showing thylakoid ultrastructure of mesophyll chloroplasts from WT and HYR under drought. Cp, chloroplast; V, vascular bundle; Sg, starch grain; P, plastoglobulus; Thy, thylakoid

FIG. 11 contains pictures showing the root phenotype of rice WT and HYR overexpressed lines. (a) Wild-type and transgenic (HYR-4) rice grown on nutrient free medium for 7 days, showing the adventitious root phenotype. (b) Graph showing the number of adventitious roots in WT and five HYR transgenic lines. Bars represent mean±SE (n=10). (c) WT and five HYR transgenic rice lines showing the root morphology. The plants are grown in sand for 30 days with the supplements of Hoagland solution. (d) Light microscope images of WT and representative HYR line (HYR-4) showing thicker root tip. (e) and (f), showing microscopic images (20×) of root tissues (sections taken 1 cm above the tip) from WT and HYR, respectively. The prominent structure of enlarged cortex, stele and epidermis are seen in HYR-4 roots (f). The inset showing cortex and epidermis regions at higher magnification (40×).

FIG. 12 is a graphical representation of the gravimetric analysis of root biomass in rice WT and HYR lines under well watered (WW) and drought (DR) stress conditions. Values are means±SE of (n=6 of each genotype). Asterisks indicate levels of significance compared with wild type (t-test; *P≦0.05, ** P≦0.01)

FIG. 13 is a graphical representation of the quantitative gene expression analysis assessed through qRT-PCR. Relative expression of (selected from MA list) putative photosynthesis and carbohydrate biosynthesis genes in HYR plants compared with that in the WT. Data are expressed In the qRT-PCR as the mean relative transcript levels in HYR compared to that of WT (log 2 ratio). Error bars represent mean±SE (n=3).

### DETAILED DESCRIPTION OF VARIOUS EMBODIMENTS OF THE INVENTION

The present invention may be understood more readily by reference to the following detailed description of the preferred embodiments of the invention and the examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

The present invention relates to a nucleic acid sequence encoding a HYR protein that are important in increasing plant root growth, and/or yield, and/or for modulating a plant's response to an environmental stress. More particularly, expression of these nucleic acids in a crop plant results in modulation (increase or decrease, preferably increase) in root growth, and/or increased yield, and/or increased tolerance to an environmental stress.

Accordingly, the present invention encompasses a transgenic crop plant comprising a nucleic acid sequence encoding a HYR protein wherein such HYR protein comprises the sequence of SEQ ID NO:1, a sequence with at least 70% similarity to SEQ ID NO:1, a sequence encoding an ortholog protein, a sequence encoding a homologous protein, a functional fragment, and any combination thereof, and methods of producing such transgenic crop plant, wherein the expression of the HYR protein in the plant results in increased root growth, and/or yield, and/or tolerance to an environmental stress as detected by enhanced water use efficiency, enhanced photosynthesis, enhanced biomass, enhanced grain yield, enhanced drought tolerance, and any combination thereof.

In one embodiment, the HYR encoding sequences are from a plant, preferably an Arabidopsis plant, a canola plant, a soybean plant, a rice plant, a barley plant, a sunflower plant, a linseed plant, a wheat plant, or a maize plant.

The present invention further encompasses novel nucleic acid sequences and their use for increasing a plant's root growth, and/or yield, and/or tolerance to an environmental stress. The present invention provides a transgenic plant transformed by an HYR encoding nucleic acid, wherein expression of the nucleic acid sequence in the plant results in increased root growth, and/or increased yield, and/or increased tolerance to an environmental stress as compared to a wild type variety of the plant. In particular, the increased root growth is an increase in the length of the roots. The term “plant” as used in this specification can, depending on the context, be understood to refer to whole plants, plant cells, and plant parts including seeds. The word “plant” also refers to any plant, particularly, to seed plant, and may include, but not limited to, crop plants. Plant parts include, but are not limited to, stems, roots, ovules, stamens, leaves, embryos, meristematic regions, callus tissue, gametophytes, sporophytes, pollen, microspores, and the like. In one embodiment, the transgenic plant is male sterile. Also provided is a plant seed or fruit produced by a transgenic plant transformed by an HYR encoding nucleic acid, wherein the seed contains the HYR encoding nucleic acid, and wherein the plant is true breeding for increased root growth, and/or increased yield, and/or increased tolerance to environmental stress as compared to a wild type variety of the plant.

The invention also provides a product produced by or from the transgenic plants expressing the HYR encoding sequences, their plant parts, or their seeds. The product can be obtained using various methods well known in the art. As used herein, the word “product” includes, but is not limited to, a foodstuff, feedstuff, a food supplement, feed supplement, cosmetic or pharmaceutical. Foodstuffs are regarded as compositions used for nutrition. These also include compositions for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

As used herein, the term “variety” refers to a group of plants within a species that share constant characters that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more DNA sequences introduced into a plant variety.

The plants according to the invention include monocotyledonous plants, such as, for example, cereals such as wheat, barley, sorghum and millet, rye, triticale, maize, rice or oats, and sugarcane. Further preferred are trees such as apple, pear, quince, plum, cherry, peach, nectarine, apricot, papaya, mango, and other woody species including coniferous and deciduous trees such as poplar, pine, sequoia, cedar, oak, etc. Especially preferred are Arabidopsis thaliana, Nicotiana tabacum, oilseed rape, soybean, corn (maize), wheat, linseed, potato and tagetes.

The transgenic crop plant according to the invention may be a plant selected from a genus of the group consisting of: Zea, Oryza, Triticum, Solanum, Hordeum, Brassica, Glycine, Phaseolus, Avena, Sorghum, Saccharum, Gossypium, Populus, Quercus, Salix, Miscanthus, Panicum, and any combination thereof.

The present invention describes for the first time that the HYR encoding nucleic acids result in better crop yield under normal and stressful environmental conditions. Homologs and orthologs of the amino acid sequences are defined below.

The HYRs of the present invention are preferably produced by recombinant DNA techniques. For example, a nucleic acid molecule encoding the polypeptide is cloned into an expression vector (as described below), the expression vector is introduced into a host cell (as described below) and the HYR is expressed in the host cell. The HYR can then be isolated from the cells by an appropriate purification scheme using standard polypeptide purification techniques. For purposes of the invention, the term “recombinant polynucleotide” refers to a polynucleotide that has been altered, rearranged, or modified by genetic engineering. Examples include any cloned polynucleotide, and polynucleotides that are linked or joined to heterologous sequences. The term “recombinant” does not refer to alterations to polynucleotides that result from naturally occurring events, such as spontaneous mutations. Alternative to recombinant expression, an HYR encoding sequence, or peptide thereof, can be synthesized chemically using standard peptide synthesis techniques. Moreover, native HYR can be isolated from cells (e.g., Arabidopsis thaliana cells), for example using an anti-HYR antibody, which can be produced by standard techniques utilizing an HYR or fragment thereof.

As used herein, the term “environmental stress” refers to sub-optimal conditions associated with salinity, drought, temperature, metal, chemical, pathogenic and oxidative stresses, or combinations thereof. In preferred embodiments, the environmental stress can be selected from one or more of the group consisting of salinity, drought, or temperature, or combinations thereof, and in particular, can be selected from one or more of the group consisting of high salinity, low water content (drought), or low temperature. In a more preferred embodiment, the environmental stress is drought stress. As also used herein, the term “water use efficiency” refers to the amount of organic matter produced by a plant divided by the amount of water used by the plant in producing it, i.e. the dry weight of a plant in relation to the plant's water use. As used herein, the term “dry weight” refers to everything in the plant other than water, and includes, for example, carbohydrates, proteins, oils, and mineral nutrients. It is also to be understood that as used in the specification and in the claims, “a” or “an” can mean one or more or at least one, depending upon the context in which it is used. Thus, for example, reference to “a cell” can mean that at least one cell can be utilized.

A nucleic acid molecule according to the present invention, e.g., a nucleic acid molecule having a nucleotide sequence as set forth in any of SEQ ID NOS as provided herein, or a portion thereof, can be isolated using standard molecular biology techniques and the sequence information provided herein. Preferred are the SEQ ID NOS specifically provided, however, other sequences may also be used especially if such sequences comprise at least 70% identity with the specified SEQ ID NO, such as 75% similarity or identity, or from about 80-90% similarity or identity, or from about 85-95% similarity/identity. Especially preferred are sequences that share from about 90%, 91%, 92%, 93%, 94%, 95%, 96%, 97%, 98%, 99% or 100% identity with the specified SEQ ID NO.

The invention also provides a nucleic acid sequence encoding a HYR protein fused or operably linked to a transcription regulatory sequence active in plant cells. As used herein, an HYR “chimeric polypeptide” or “fusion polypeptide” comprises a HYR operatively linked to a transcription regulatory sequence. With respect to the fusion polypeptide, the term “operatively linked” is intended to indicate that the HYR and the transcription regulatory protein are fused to each other so that both proteins fulfill the proposed function attributed to the sequence used. The transcription regulatory protein can be fused to the N-terminus or C-terminus of the HYR.

Preferably, an HYR or fusion polypeptide of the invention is produced by standard recombinant DNA techniques. For example, DNA fragments coding for the different polypeptide sequences can be ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (See, for example, Current Protocols in Molecular Biology, Eds. Ausubel et al. John Wiley & Sons: 1992). Moreover, many expression vectors are commercially available that already encode a fusion moiety (e.g., a GST polypeptide). An HYR encoding nucleic acid can be cloned into such an expression vector such that the fusion moiety is linked in-frame to the HYR.

In addition to fragments and fusion polypeptides of the HYRs described herein, the present invention includes homologs and analogs of naturally occurring HYRs and HYR encoding nucleic acids in a plant. “Homologs” in the context of this specification are defined as two nucleic acids or polypeptides that have similar, or identical, nucleotide or amino acid sequences, respectively. Homologs include allelic variants, orthologs, paralogs, agonists, and antagonists of HYRs as defined hereafter. As used herein, a “naturally occurring” HYR refers to a HYR amino acid sequence that occurs in nature.

As stated above, the present invention relates to HYRs, sequences with at least 70% identity to HYR, and homologs thereof. To determine the percent sequence identity of two amino acid sequences, the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence, then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions.times.100). Preferably, the isolated amino acid homologs included in the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence shown in any of SEQ ID NOS herein. In yet another embodiment, the isolated amino acid homologs included in the present invention are at least about 70%, preferably at least about 70-80%, and more preferably at least about 70-75%, 75-80%, 80-85%, 85-90%, or 90-95%, and most preferably at least about 96%, 97%, 98%, 99%, or more identical to an entire amino acid sequence encoded by a nucleic acid sequence shown in any of SEQ ID NOS as provided herein.

Moreover, nucleic acid molecules encoding HYRs from the same or other species such as HYR analogs, orthologs, and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, R. L. et al., 1997, Science 278(5338):631-637). Analogs; orthologs, and paralogs of a naturally occurring HYR can differ from the naturally occurring HYR by post-translational modifications, by amino acid sequence differences, or by both. Post-translational modifications include in vivo and in vitro chemical derivatization of polypeptides, e.g., acetylation, carboxylation, phosphorylation, or glycosylation, and such modifications may occur during polypeptide synthesis or processing or following treatment with isolated modifying enzymes.

The invention further provides an isolated recombinant expression vector comprising an HYR encoding nucleic acid and a tissue specific-, inducible- or developmentally regulated promoter active in plant cells, wherein expression of the vector in a host cell results in increased tolerance to environmental stress as compared to a wild type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. As used herein with respect to a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel, Gene Expression Technology Methods in Enzymology 185, Academic Press, San Diego, Calif. (1990) and Gruber and Crosby, in: Methods in Plant Molecular Biology and Biotechnology, eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein.

The recombinant expression vectors of the invention can be designed for expression of HYRs in prokaryotic or eukaryotic cells. For example, HYR genes can be expressed in bacterial cells such as C. glutamicum, insect cells (using baculovirus expression vectors), yeast and other fungal cells (See Romanos, M. A. et al., 1992, Foreign gene expression in yeast: a review, Yeast 8:423-488; van den Hondel, C. A. M. J. J. et al., 1991, Heterologous gene expression in filamentous fungi, in: More Gene Manipulations in Fungi, J. W. Bennet & L. L. Lasure, eds., p. 396-428: Academic Press: San Diego; and van den Hondel, C. A. M. J. J. & Punt, P. J., 1991, Gene transfer systems and vector development for filamentous fungi, in: Applied Molecular Genetics of Fungi, Peberdy, J. F. et al., eds., p. 1-28, Cambridge University Press: Cambridge), algae (Falciatore et al., 1999, Marine Biotechnology 1(3):239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in PCT Application No. WO 98/01572, and multicellular plant cells (See Schmidt, R. and Willmitzer, L., 1988, High efficiency Agrobacterium tumefaciens-mediated transformation of Arabidopsis thaliana leaf and cotyledon explants, Plant Cell Rep. 583-586; Plant Molecular Biology and Biotechnology, C Press, Boca Raton, Fla., chapter 6/7, S.71-119 (1993); F. F. White, B. Jenes et al., Techniques for Gene Transfer, in: Transgenic Plants, Vol. 1, Engineering and Utilization, eds. Kung and R. Wu, 128-43, Academic Press: 1993; Pottykus, 1991, Annu. Rev. Plant Physiol. Plant Molec. Biol. 42:205-225 and references cited therein), or mammalian cells. Suitable host cells are discussed further in Goeddel, Gene Expression Technology Methods in Enzymology 185, Academic Press: San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide but also to the C-terminus or fused within suitable regions in the polypeptides. Such fusion vectors typically serve three purposes: 1) to increase expression of a recombinant polypeptide; 2) to increase the solubility of a recombinant polypeptide; and 3) to aid in the purification of a recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin, and enterokinase.

Typical fusion expression vectors include pGEX (Pharmacia Biotech Inc; Smith, D. B. and Johnson, K. S., 1988, Gene 67:31-40), pMAL (New England Biolabs, Beverly, Mass.), and pRIT5 (Pharmacia, Piscataway, N.J.) which fuse glutathione S-transferase (GST), maltose E binding polypeptide, or polypeptide A, respectively, to the target recombinant polypeptide. In one embodiment, the coding sequence of the HYR is cloned into a pGEX expression vector to create a vector encoding a fusion polypeptide comprising, from the N-terminus to the C-terminus, GST-thrombin cleavage site-X polypeptide. The fusion polypeptide can be purified by affinity chromatography using glutathione-agarose resin. Recombinant HYR unfused to GST can be recovered by cleavage of the fusion polypeptide with thrombin.

Examples of suitable inducible non-fusion E. coli expression vectors include pTrc (Amann et al., 1988, Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant polypeptide expression is to express the polypeptide in a host bacterium with an impaired capacity to proteolytically cleave the recombinant polypeptide (Gottesman, S., Gene Expression Technology: Methods in Enzymology 185, Academic Press, San Diego, Calif. (1990) 119-128). Another strategy is to alter the sequence of the nucleic acid to be inserted into an expression vector so that the individual codons for each amino acid are those preferentially utilized in the bacterium chosen for expression, such as C. glutamicum (Wada et al., 1992, Nucleic Acids Res. 20:2111-2118). Such alteration of nucleic acid sequences of the invention can be carried out by standard DNA synthesis techniques.

In another embodiment, the HYR expression vector is a yeast expression vector. Examples of vectors for expression in yeast S. cerevisiae include pYepSec1 (Baldari, et al., 1987, EMBO J. 6:229-234), pMFa (Kurjan and Herskowitz, 1982, Cell 30:933-943), pJRY88 (Schultz et al., 1987, Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J., 1991, “Gene transfer systems and vector development for filamentous fungi,” in: Applied Molecular Genetics of Fungi, J. F. Peberdy, et al., eds., p. 1-28, Cambridge University Press: Cambridge.

In a preferred embodiment of the present invention, the HYRs are expressed in plants and plants cells such as unicellular plant cells (e.g. algae) (See Falciatore et al., 1999, Marine Biotechnology 1(3):239-251 and references therein) and plant cells from higher plants (e.g., the spermatophytes, such as crop plants). An HYR may be introduced or incorporated into a plant cell by any means, including transfection, transformation or transduction, electroporation, particle bombardment, agroinfection, and the like.

Other suitable methods for transforming or transfecting host cells including plant cells can be found in Sambrook, et al. (Molecular Cloning: A Laboratory Manual. latest ed., Cold Spring Harbor Laboratory, Cold Spring Harbor Laboratory Press, Cold Spring Harbor, N.Y., 1989) and other laboratory manuals such as Methods in Molecular Biology, 1995, Vol. 44, Agrobacterium protocols, ed: Gartland and Davey, Humana Press, Totowa, N.J. As increased growth and increased biotic and abiotic stress tolerance are general traits wished to be inherited into a wide variety of plants like maize, wheat, rye, oat, triticale, rice, barley, sorghum, millet, sugarcane, soybean, peanut, cotton, rapeseed and canola, manihot, pepper, sunflower and tagetes, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. Forage crops include, but are not limited to, Wheatgrass, Canarygrass, Bromegrass, Wildrye Grass, Bluegrass, Orchardgrass, Alfalfa, Salfoin, Birdsfoot Trefoil, Alsike Clover, Red Clover, and Sweet Clover.

According to the present invention, the introduced HYR may be maintained in the plant cell stably if it is incorporated into a non-chromosomal autonomous replicon or integrated into the plant chromosomes. Alternatively, the introduced HYR may be present on an extra-chromosomal non-replicating vector and may be transiently expressed or transiently active.

The promoter may be constitutive, inducible, developmental stage-preferred, cell type-preferred, tissue-preferred, or organ-preferred. Constitutive promoters are active under most conditions. Examples of constitutive promoters include the CaMV 19S and 35S promoters (Odell et al., 1985, Nature 313:810-812), the sX CaMV 35S promoter (Kay et al., 1987, Science 236:1299-1302) the Sep1 promoter, the rice actin promoter (McElroy et al., 1990, Plant Cell 2:163-171), the Arabidopsis actin promoter, the ubiquitan promoter (Christensen et al., 1989, Plant Molec. Biol. 18:675-689), pEmu (Last et al., 1991, Theor. Appl. Genet. 81:581-588), the figwort mosaic virus 35S promoter, the Smas promoter (Velten et al., 1984, EMBO J. 3:2723-2730), the super-promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

Inducible promoters are preferentially active under certain environmental conditions, such as the presence or absence of a nutrient or metabolite, heat or cold, light, pathogen attack, anaerobic conditions, and the like. For example, the hsp80 promoter from Brassica is induced by heat shock; the PPDK promoter is induced by light; the PR-1 promoter from tobacco, Arabidopsis, and maize are inducible by infection with a pathogen; and the Adh1 promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if gene expression is wanted to occur in a time specific manner. Examples of such promoters are a salicylic acid inducible promoter (PCT Published Application No. WO 95/19443), a tetracycline inducible promoter (Gatz et al., 1992, Plant J. 2:397-404), and an ethanol inducible promoter (PCT Published Application No. WO 93/21334).

In one preferred embodiment of the present invention, the inducible promoter is a stress-inducible promoter. For the purposes of the invention, stress inducible promoters are preferentially active under one or more of the following stresses: sub-optimal conditions associated with salinity, drought, temperature, metal, chemical, pathogenic, and oxidative stresses. Stress inducible promoters include, but are not limited to, Cor78 (Chak et al., 2000, Planta 210:875-883; Hovath et al., 1993, Plant Physiol. 103:1047-1053), Cor15a (Artus et al., 1996, PNAS 93(23):13404-09), Rci2A (Medina et al., 2001, Plant Physiol. 125:1655-66; Nylander et al., 2001, Plant Mol. Biol. 45:341-52; Navarre and Goffeau, 2000, EMBO J. 19:2515-24; Capel et al., 1997, Plant Physiol. 115:569-76), Rd22 (Xiong et al., 2001, Plant Cell 13:2063-83; Abe et al., 1997, Plant Cell 9:1859-68; Iwasaki et al., 1995, Mol. Gen. Genet. 247:391-8), cDet6 (Lang and Palve, 1992, Plant Mol. Biol. 20:951-62), ADH1 (Hoeren et al., 1998, Genetics 149:479-90), KAT1 (Nakamura et al., 1995, Plant Physiol. 109:371-4), KST1 (Muller-Rober et al., 1995, EMBO 14:2409-16), Rha1 (Terryn et al., 1993, Plant Cell 5:1761-9; Terryn et al., 1992, FEBS Lett. 299(3):287-90), ARSK1 (Atkinson et al., 1997, GenBank Accession # L22302, and PCT Application No. WO 97/20057), PtxA (Plesch et al., GenBank Accession # X67427), SbHRGP3 (Alm et al., 1996, Plant Cell 8:1477-90), GH3 (Liu et al., 1994, Plant Cell 6:645-57), the pathogen inducible PRP1-gene promoter (Ward et al., 1993, Plant. Mol. Biol. 22:361-366), the heat inducible hsp80-promoter from tomato (U.S. Pat. No. 5,187,267), cold inducible alpha-amylase promoter from potato (PCT Application No. WO 96/12814), or the wound-inducible pinII-promoter (European Patent No. 375091). For other examples of drought, cold, and salt-inducible promoters, such as the RD29A promoter, see Yamaguchi-Shinozalei et al., 1993, Mol. Gen. Genet. 236:331-340.

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters, and the like. Seed preferred promoters are preferentially expressed during seed development and/or germination. For example, seed preferred promoters can be embryo-preferred, endosperm preferred, and seed coat-preferred. See Thompson et al., 1989, BioEssays 10:108. Examples of seed preferred promoters include, but are not limited to, cellulose synthase (celA), Cim1, gamma-zein, globulin-1, maize 19 kD zein (cZ19B1), and the like.

Other suitable tissue-preferred or organ-preferred promoters include the napin-gene promoter from rapeseed (U.S. Pat. No. 5,608,152), the USP-promoter from Vicia faba (Baeumlein et al., 1991, Mol. Gen. Genet. 225(3):459-67), the oleosin-promoter from Arabidopsis (PCT Published Application No. WO 98/45461), the phaseolin-promoter from Phaseolus vulgaris (U.S. Pat. No. 5,504,200), the Bce-4-promoter from Brassica (PCT Published Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the Ipt2 or Ipt1-gene promoter from barley (PCT Published Application No. WO 95/15389 and PCT Published Application No. WO 95/23230) or those described in PCT Published Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the .beta.-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

Additional flexibility in controlling heterologous gene expression in plants may be obtained by using DNA binding domains and response elements from heterologous sources (i.e., DNA binding domains from non-plant sources). An example of such a heterologous DNA binding domain is the LexA DNA binding domain (Brent and Ptashne, 1985, Cell 43:729-736).

Another aspect of the invention pertains to host cells into which a recombinant expression vector of the invention has been introduced. The terms “host cell” and “recombinant host cell” are used interchangeably herein. It is understood that such terms refer not only to the particular subject cell but they also apply to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell.

A host cell of the invention, such as a prokaryotic or eukaryotic host cell in culture, can be used to produce (i.e., express) an HYR. Accordingly, the invention further provides methods for producing HYRs using the host cells of the invention.

The HYR nucleic acid molecules according to the invention have a variety of uses. Most importantly, the nucleic acid and amino acid sequences of the present invention can be used to transform plants, particularly crop plants, thereby inducing tolerance to stresses such as drought, high salinity, and cold. The present invention therefore provides a transgenic plant transformed by an HYR encoding nucleic acid, wherein expression of the nucleic acid sequence in the plant results in increased tolerance to environmental stress as compared to a wild type variety of the plant and higher yield. The transgenic plant can be a monocot or a dicot. The invention further provides that the transgenic plant can be selected from maize, wheat, rye, oat, triticale, rice, barley, sorghum, millet, sugarcane, soybean, peanut, cotton, rapeseed, canola, manihot, pepper, sunflower, tagetes, solanaceous plants, potato, tobacco, eggplant, tomato, Vicia species, pea, alfalfa, coffee, cacao, tea, Salix species, oil palm, coconut, perennial grass, and forage crops, for example.

To facilitate a better understanding of the present invention, the following examples of certain aspects of some embodiments are given. In no way should the following examples be read to limit, or define, the scope of the invention.

The following non-limiting Examples exemplify the use of HYR genes for modifying plant phenotypes. Unless stated otherwise, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R.D.D. Croy, jointly published by BIOS Scientific Publications Ltd (UK) and Blackwell Scientific Publications, U K.

### EXAMPLES

### Example 1

### Generation and Selection of Rice Transformants with the HYR Gene

The HYR Transcription Factor is a Drought-Induced Gene in Rice Reproductive Tissues.

The rice genome is predicted to contain 139 AP2/ERF domain containing transcription factor genes (Nakano et al., 2006). To identify stress-inducible AP2 genes a rice drought microarray experiment was conducted, which identified Os03g02650 termed OsHYR as one of the novel transcription factors (TF) induced by drought at critical reproductive developmental stage, known as pre-anthesis. Further, to confirm whether OsHYR is part of a native drought-response pathway, quantitative RT-PCR was used to determine the expression profile of the gene at different developmental stages; including two critical reproductive phases: pre-anthesis stage (end of booting stage, panicle elongation) and post-anthesis stage (2 weeks after flowering) by withholding water for a period of 4-8 days of progressive drought.

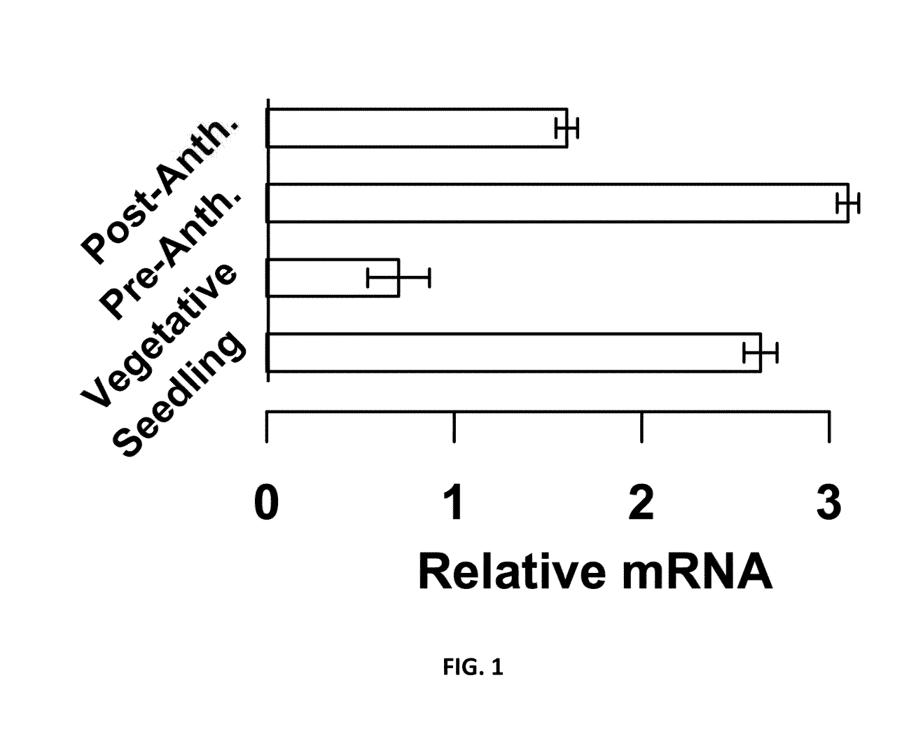

As shown in FIG. 1, OsHYR transcript levels are predominantly expressed in panicles about 3 fold at pre-anthesis and 1.5 fold at post-anthesis under severe drought relative to well watered conditions. Thus, OsHYR is a key regulator, demonstrating the potential expression at yield-sensitive critical reproductive stages.

An important part of stress responses is the differential regulation of the plant transcriptome by TF, which regulate the temporal and spatial expression patterns of specific genes. Previous experiments have shown that genes with putative functions in drought include NAM, HLH, G-box, Zinc finger, and AP2 TF (Zhou et al., 2007). Most of the AP2/ERF TF whose transcription properties have been studied are activators of transcription, although some are repressors (Fujimoto et al., 2000). The AP2/ERF family proteins have a DRE cis-element binding motif, believed to be involved in the expression of dehydration-responsive genes. Homologs of the protein under study have been found to respond to dehydration or drought stress (Fujimoto et al., 2000; Haake et al., 2002) or function in drought responses, such as DREB2A (Sakuma et al., 2006). Thus, it is no surprise that the TF identified responded to drought stress.

Generation of Rice Transformants Overexpressing the HYR Transcription Factor.

To make an overexpression construct of the gene, the full-length cDNA of Os03g02650/OsHYR was amplified (using pfu DNA polymerase) from genomic DNA of rice cv. Nipponbare using oligonucleotides OsHYR_F (5′-GTGTTCGAGATGGATCGAGAC-3′ (SEQ ID NO:5)) and OsHYR_R (5′-GCCCATTTCAGGAATGGTTCCAC-3′(SEQ ID NO:6)). The amplified 619 bp OsHYR fragment was cloned behind the 35S Cauliflower Mosaic Virus (CaMV35S) promoter for constitutive expression, generating the 35S:HYR vector.

Transformation Experiment:

The CaMV35S-OsHYR construct was then introduced into rice (cultivar Nipponbare) by Agrobacterium-mediated transformation (Hei et al., 1994; Nishimura et al., 2007). Briefly mature seeds were dehusked and sterilized in 70% (vol/vol) ethanol for 1-2 minutes and then transferred to 50% (vol/vol) chlorox solution for 30 minutes with gentle shaking. The seeds were rinsed 5 times with sterile water. The sterilized seeds were then plated for callus induction on Murashige and Skoog (MS) medium supplemented with 3 mg/L 2,4-dichlorophenoxyacetic acid (2,4-D)/0.3 g/L casamino acid/30 g/L sucrose/3 g/L proline/0.1 g/L myo-inositol/3 g/L gellan gum, pH 5.8 (MSCI) and grown for 21-28 days. Three-four weeks after callus induction from the scutellar region of the rice embryo, calli were immersed in Agrobacterium tumefaciens suspension for 10 minutes with gentle shaking to infect them. Infected calli were co-cultivated with Agrobacterium in MSCI supplemented with 0.5 g/L casamino acid/100 μM acetosyringone/68.5 g/L sucrose/36 g g/L glucose/0.9 g/L L-glutamine/0.3 g/L L-aspartic acid/3 g/L potassium chloride, PH 5.2 (MSCC). After 3 d of co-cultivation, calli were washed 5 times with sterile water followed by 225 mg/L cefotaxime and further with 250 mg/L carbenicillin. The calli were rinsed 3 times with sterile water to wash off the antibiotics and blotted on sterile filter paper. The calli were plated on MSCI supplemented with 50 mg/L hygromycin/225 mg/L cefotaxime/250 mg/L carbinicillin/3 g/L gellan gum, pH 5.8 (MSSE) and incubated in a tissue culture growth chamber at 28° C. and 250 μmol m⁻² s⁻¹. The calli were subcultured on MSSE every two weeks until plant regeneration was observed. The MS tissue culture media, its supplements and antibiotics were sourced from Sigma, St. Louis, Mo. The regenerated plantlets were grown on MS media supplemented with 0.1 g/L myo-inositol/30 g/L sucrose/100×MS vitamins, pH 5.8 (MSPG) in a tissue culture growth chamber at 28° C., 250 μmol m⁻² s⁻¹, 16-h light/8-h dark period for 2-3 weeks. The regenerated plantlets (putative primary transformants; T0) were transplanted into pots and grown in environmental controlled plant growth chamber for another 3 weeks and transferred to the greenhouse and grown further to maturity.

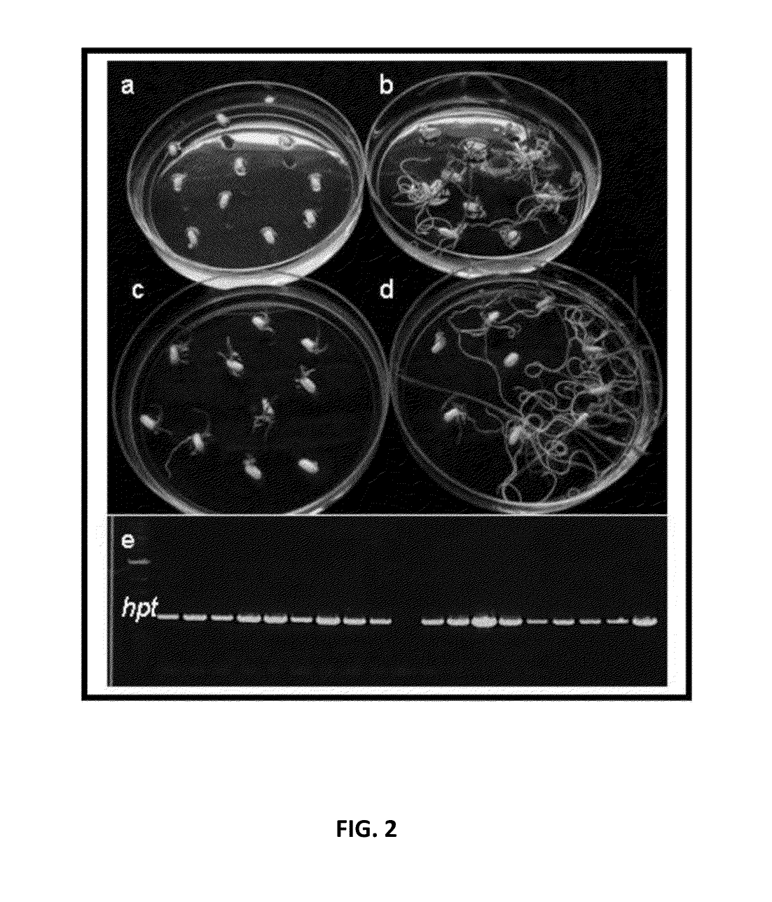

Seeds from the putative transformants were tested for hygromycin resistance by germinating them on 50 mg l⁻¹ hygromycin (Sigma, Saint Louis Mo.), following the procedures of Nishimura et al. (2007). Five hygromycin-resistant lines (HYR-2, HYR-4, HYR-12, HYR-16, and HYR-45) were identified (FIGS. 2 a-d). Seeds from the hygromycin-resistant seed lots were planted, and individual plants were genotyped by PCR using primers to amplify the hygromycin phosphotransferase (hpt) gene marker. DNA was isolated from 3 week old seedlings. About 3-5 cm leaf materials was ground in liquid nitrogen. 400 μL 1× isolation and 400 μL phenol:chloroform was added and the material was gently shaken manually for 5 min. The supernatant was removed into fresh tubes and 250 μL isopropanol was added, mixed and DNA was precipitated for 5 min at room temperature, followed by centrifuging at 7500 rpm for 20 min. The isopropanol was discarded and the pellet dried at room temperature for 1-h. The pellet was washed by adding 200 μL of 70% ethanol and centrifuging for 5 min at 7500 rpm. The ethanol was poured of and the pellet dried for 1-h at room temperature. The DNA pellet was dissolved in 50 μL+10 μg/ml RNase A. The DNA was used template for PCR analysis The primer sequence used was; hpt forward, CGATTGCGTCGCATCGACCCTGCGC (SEQ ID NO:7) and hpt reverse, CGACCTGATGCAGCTCTCCGAGGGC (SEQ ID NO:8). PCR was carried out with Taq DNA polymerase (New England Biolabs Inc., Ipswich, Mass.) in 20 μL reaction volume in a thermal cycler (IQ5, Bio-Rad Laboratories, Hercules, Calif.). The PCR cycle program consisted of initial denaturation at 95° C. for 3 min, followed by 30 cycles of denaturation at 95° C. for 1 min, annealing at 55° C. for 30 s, and extension at 72° C. for 1 min, followed by a final extension at 72° C. for 5 min. The PCR product was resolved by 0.8% agarose gel electrophoresis in 1×Tris-acetate-EDTA (TAE) buffer along with 1-kbp DNA ladder as marker (New England Biolabs Inc., Ipswich, Mass.). The hpt gene positive plants (FIG. 2e) were used for experiments.

Selection of Rice Transformants with the HYR Transcription Factor.

Seeds (T1 progeny) from the putative transformants were tested for germination in hygromycin, and five hygromycin-resistant lines (HYR-2, HYR-4, HYR-12, HYR-16, and HYR-45) were identified (FIGS. 2 a-d). Segregation pattern of the hygromycin resistant gene is shown in Table 1 below. The transgenic locus is hemizygous and as the transgene provided a gain-of-function phenotype (hygromycin resistance), the segregation of 3:1 Mendelian ratio for the transgenic to wild type was expected in the T1 progeny. While some genotypes were below or above this ratio, on average inheritance of the hygromycin resistant gene was 3:1, which is the expected inheritance pattern of the transgene (FIG. 2e). The transgenic rice lines that were confirmed positive to hpt gene amplification were further subjected to qRT-PCR amplification of OsHYR with gene specific forward primer (5′-CTCAACTTCCCAAACTCAG-3′ (SEQ ID NO:9)) and reverse primer (5′-CCATAACAATCGCATCCCTAG-3′(SEQ ID NO:10)) at an annealing temperature of 53° C. Based on the results of qRT-PCR amplification of OsHYR, it was evident that five lines showed significant and stable expression, and were used for further analysis described in this study.

Seeds from putative transformants were tested for dehusked and germinated in Murashige and Skog media supplemented with 50 mg/L hygromycin. Ten seeds were used for an experiment and this was repeated. Seeds that germinated and grew on hygromycin supplemented media were scored as resistance (FIG. 1), with n=20. Results are in Table 1.

TABLE 1
Segregation of hygromycin resistance gene
in self progeny of HYR-transgenic plants

| | Seed | Number | Number | hpt |
|---|---|---|---|---|
| | Number | Hygromycin | Hygromycin | Segregation |
| Genotype | Tested | Resistant | Sensitive | Ratio |
| WT | 20 | 0 | 0 | 0 |
| HYR-2 | 20 | 15 | 8 | 3.1 |
| HYR-4 | 20 | 15 | 5 | 3:1 |
| HYR-12 | 20 | 14 | 6 | 2.3:1 |
| HYR-16 | 20 | 16 | 4 | 3.5:1 |
| HYR-45 | 20 | 15 | 5 | 3:1 |

### Example 2

### Water Use Efficiency of HYR Lines

Drought Experiment:

250-ml pots were filled with a 1:1 mix of topsoil and compost (Scott-Sierra Horticultural Product Co, Marysville, Ohio). The compost was made from sphagnum/peat moss, vermiculite, and bark ash. Soil at near field capacity was weighed and filled into tared pots. One-week old seedlings of HYR and wild type (WT) rice were transplanted into the pots (one plant per pot). The pots were placed in water-filled trays to simulate flooded/paddy conditions and supplied with a general purpose 20-20-20 fertilizer (Scott-Sierra Horticultural Product Co, Marysville, Ohio) dissolved in water to provide 50 kg N, P₂O₅, and K₂O ha⁻¹. Fertilizer was applied once a week throughout the growing period. Twenty-eight days after planting (DAP), the pots were adjusted to equal weights (soil+water) by adding water as needed, and they were mulched with a layer of perlite of fixed weight to minimize evaporative water loss from the soil surface. The pots were removed from the water-filled trays and placed on tared bases. Sixteen pots of each genotype were divided in two, with eight for drought-stress treatment and eight for well-watered controls. The drought-stress treatments involved withholding irrigation and drying down to approximately 70% field capacity, which took 3 days. At this time (31 DAP), shoots on half of the plants (four for drought and four for control treatment) for each line were harvested and dried at 72° C. for 96 hr and this biomass was designated as BIO-31.

For the remaining plants, gravimetric soil moisture was maintained at 70% (drought-stressed) or essentially at field capacity (well-watered) by replacing water lost through transpiration. This was done at mid-morning and late afternoon. The amount of water added each day for each pot was noted for calculation of cumulative water used (WUc). This watering regime was continued for 14 d. At that point (45 DAP), shoots were harvested and dried at 72° C. for 96 hr, and this biomass was designated BIO-45. WUc was calculated using the formulae: daily WU=(Total weight)−(tared pot+tared base+tared mulch), and WUc=Σ{daily WU} over 14 d. Gravimetric water use efficiency (WUEg) was calculated as: WUEg=[(BIO-45)−(BIO-31)]/(WUc). The experiment was a 2×6 factorial, with factors being watering regime (drought-exposed or well-watered) and genotype (five HYR genotypes plus WT) arranged in a completely randomized design (CRD) and replicated four times. Data were subjected to analysis of variance (ANOVA) using the general linear model (Proc GLM) of Statistical Analysis System (SAS). Differences between means were tested by the Least Significance Difference with a 0.05 threshold (LSD_{0.05}).

Gravimetric Water Use and Water Use Efficiency Measurements.

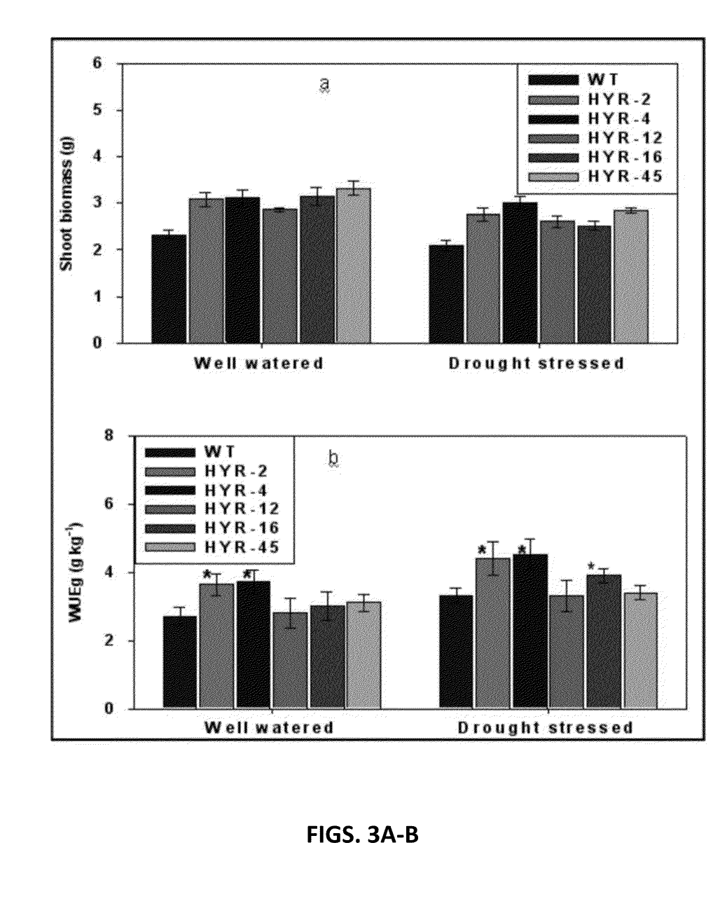

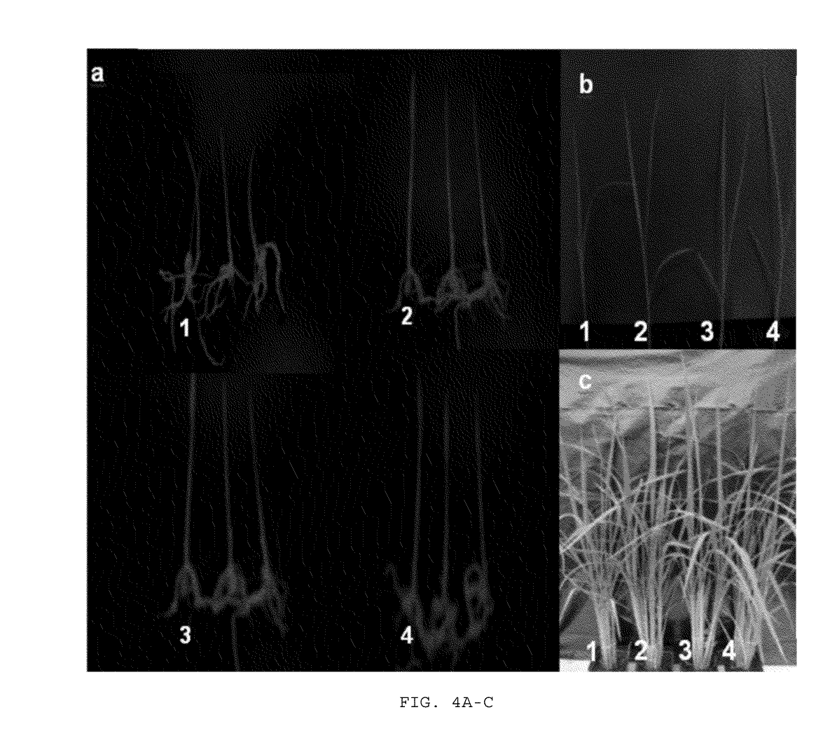

One-week-old hpt-positive seedlings, progeny of five transformants (HYR-2, HYR-4, HYR-12, HYR-16, and HYR-45) expressing the HYR gene, and WT seedlings were transplanted into pots and grown under well-watered conditions till 28 DAP. The pots from each genotype were divided into two equal sets, a set for drought-stress treatment, and the other as well-watered controls. Plants from each watering regime were harvested at 31 and 45 DAP for biomass. Their cumulative water use (WUc) and gravimetric water use efficiency (WUEg) for the 14-d interval were calculated as described. Analysis of variance (ANOVA) showed no interaction between drought stress and genotype for shoot biomass, cumulative water use (WUc), or gravimetric water use efficiency (WUEg)—the latter two parameters determined specifically for the period from 31 to 45 DAP. There were genotypic differences for shoot biomass and WUEg, but not for WUc. Drought stress reduced biomass and WUc, and consequently increased WUEg. The HYR rice lines developed higher shoot biomass than WT under both well-watered and drought-stressed conditions (FIG. 3a). In the longer-running, continuously watered experiment, the higher shoot biomass was evident as noticeably larger plants at different growth stages, which in older plants also led to more tillering (FIG. 4). However, only two lines (HYR-2 and HYR-4) had higher WUEg under both well-watered and drought conditions, while HYR-16 had increased WUEg only under drought stress (FIG. 3b).

### Example 3

### Gas Exchange Measurements of HYR Lines

Experiment:

The transgenic and WT plants were grown in tared pots under well-watered/semi-flooded conditions for 8 weeks following procedures as in Example 2. At that point, half of the plants from each genotype were allowed to dry down for 7 d until plants showed drought stress symptoms but not leaf rolling. A day before gas exchange measurements, the soil moisture in the pots with drought stress was adjusted to 75% of field capacity. Net photosynthesis (Pn), stomatal conductance (Gs), and transpiration rate (E) were measured on the youngest fully expanded leaves (one per pot) with a portable photosynthesis system LI-COR 6400 (LI-COR Inc. Lincoln, Nebr., USA). The measurements were taken between 10:30 a.m. and noon. An Arabidopsis leaf chamber (LI-COR) was used for gas exchange measurements. It provided an irradiance of 400 μmol m⁻² s⁻¹ photosynthetically active radiation (PAR), a temperature of 25 to 28° C., a CO₂ concentration of 400 μl l⁻¹, an air flow rate of 400 μmol s⁻¹, and a relative humidity of 55 to 60%. After the gas exchange measurements, soil moisture content was measured with a Delta-T theta soil moisture probe (ML2X, Dynamax, Houston, Tex.). Instantaneous water use efficiency (WUEi) was calculated using the formula: WUEi=(Pn/E) (Martin and Thorstensen, 1988). Plant water status was monitored by determination of relative water content (RWC %) according to Smart and Bingham (1974). Briefly, the same leaf used for photosynthesis measurement was excised, and an approximately 6-cm section had its fresh weight (FW) determined immediately. The leaf sections were then floated in deionized water at room temperature for 6 h, and their rehydrated weight (RW) was determined. Finally, they were dried in an oven at 70° C. overnight and weighed to obtain the dry weight (DW). The RWC % was calculated as: RWC %=(FW−DW)/(RW−DW)×100.

The experiment was a 2×4 factorial with factors being watering regime (drought stressed or well-watered) and genotype (three HYR types and WT) arranged as a completely randomized design (CRD) with four replications. Two-way analysis of variance (ANOVA) to test for the effects of drought, genotype, and their interactions was conducted using the general linear model (Proc GLM) of Statistical Analysis System (SAS). Differences between means were tested by the Least Significance Difference with a 0.05 threshold (LSD_{0.05}).

Gas Exchange Measurements of three selected rice HYR lines.

Three lines (HYR-2, HYR-4 and HYR-16) with higher biomass and WUE based on gravimetric analyses were selected for further physiological studies. In one study, gas exchange measurements were made with a LI-COR 6400 portable photosynthesis system on 8-week old plants exposed or not to 7 d of withholding irrigation. At the time of measurement, the average soil moisture content was 0.43 cm³ cm⁻³ in the well-watered controls and 0.14 cm³ cm⁻³ in the drought-stress treatments.

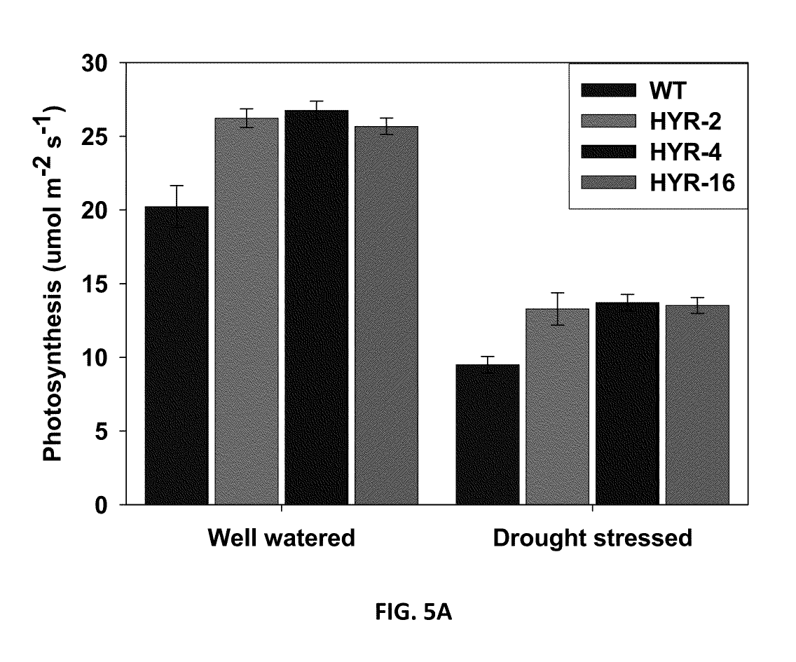

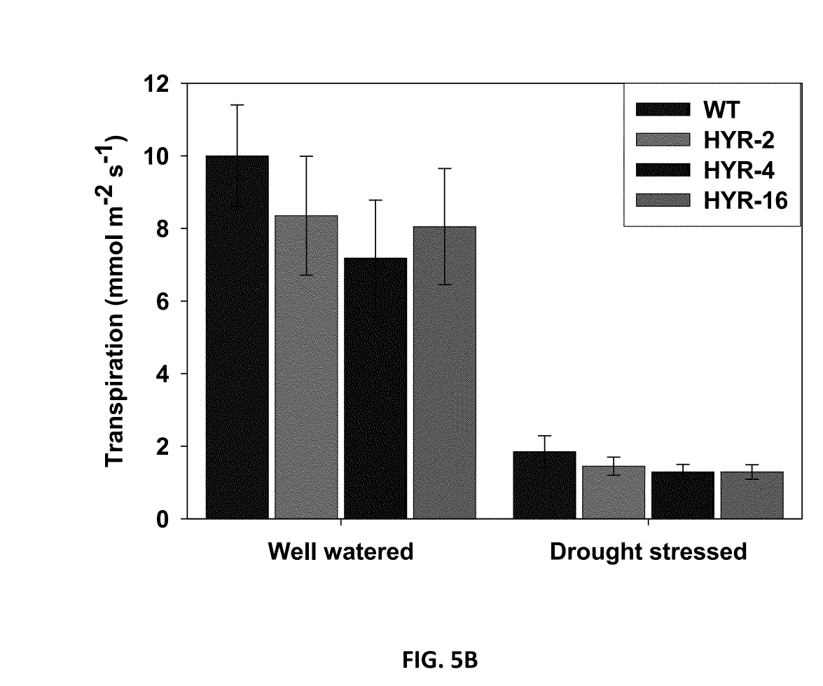

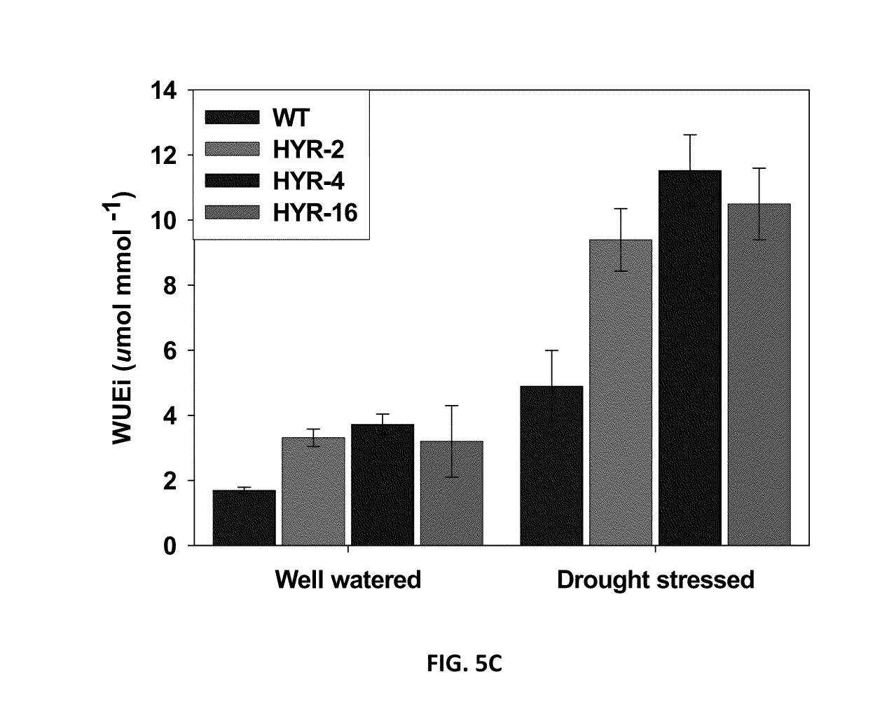

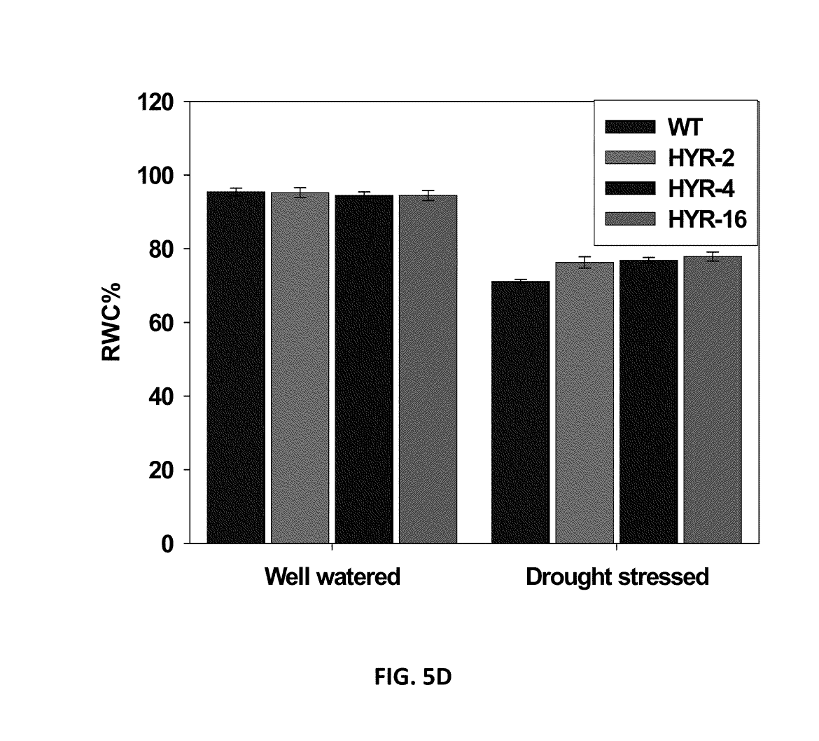

The gas exchange measurements (Pn and E) were determined and WUEi was calculated. The ANOVA revealed no interactions between drought and genotype for Pn, Gs (stomatal conductance), E, or WUEi (data not shown). Drought stress reduced Pn, Gs, and E and increased WUEi. There were genotypic differences for Pn and WUEi, while there were no genotypic differences for Gs and E. The HYR lines had higher Pn and WUEi under both watering regimes (FIG. 5), while Gs and E were not affected by soil moisture content (at the two levels tested). This suggests that WUEi is increased by an increase in Pn. The drought-stressed HYR plants had higher RWC % relative to the WT, whereas the soil moisture content was not significantly different. At equal soil moisture content, the transgenic plants maintained higher Pn, WUEi, and RWC % compared to WT.

Gas Exchange, RWC Measurements and Drought Response of Five Rice HYR Lines.

In addition to the three lines (HYR-2, HYR-4, and HYR-16) exhibiting higher biomass production and greater WUE in the previous experiment, all five lines (HYR-2, HYR-4, HYR-12, HYR-16 and HYR-45) were evaluated again with 6 replications for further in depth physiological analysis. The transgenic and WT plants were grown side by side to the late vegetative stage in tared pots under well-watered/semi-flooded conditions for 8 wks as outlined above. At that point, one-half of the plants from each genotype along with WT were allowed to dry down for 4-8 d until plants showed drought stress symptoms but not leaf rolling. A day before gas exchange measurements, the soil moisture in the pots with drought stress was adjusted to 75% of field capacity. Net photosynthesis (Pn), stomatal conductance (Gs), and transpiration rate (E) were measured on the youngest fully expanded leaves (one per pot) with a portable photosynthesis system LI-COR 6400 (LI-COR Inc. Lincoln, Nebr., USA), taken between 10:30 a.m. and noon.

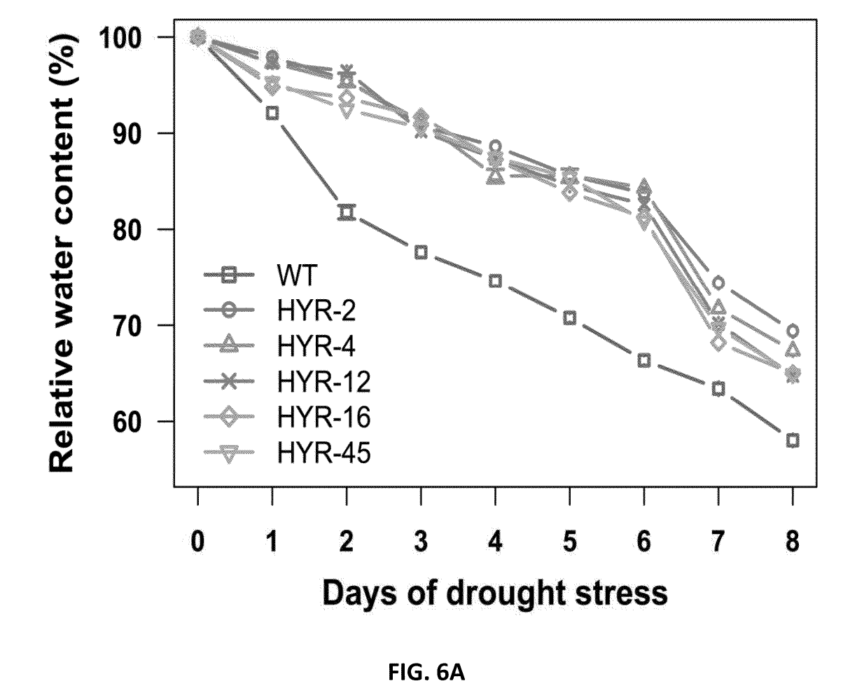

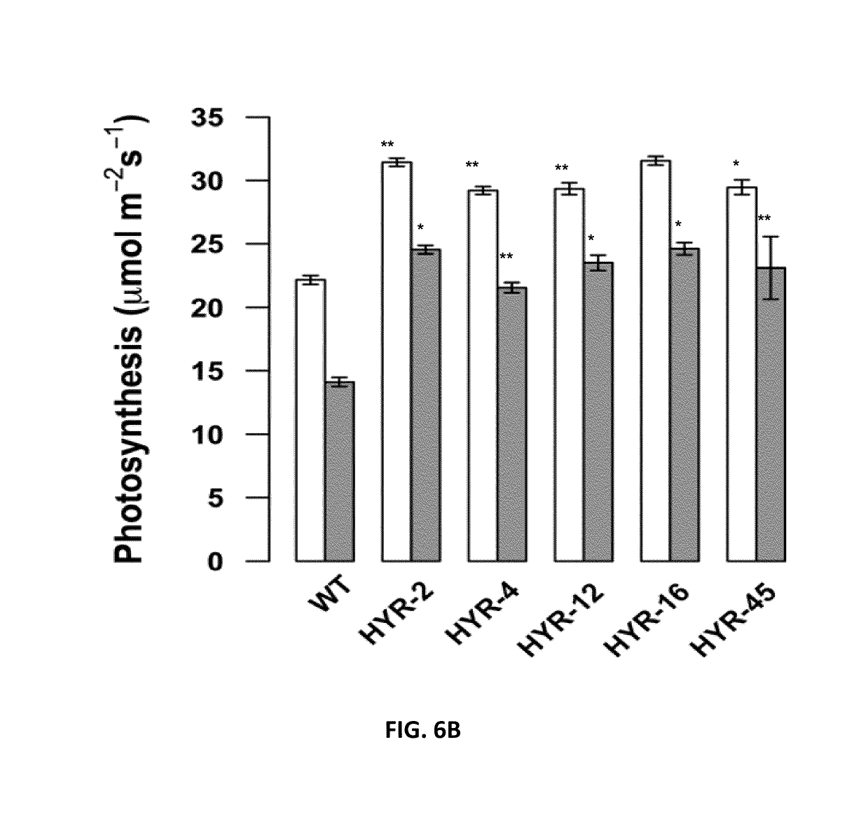

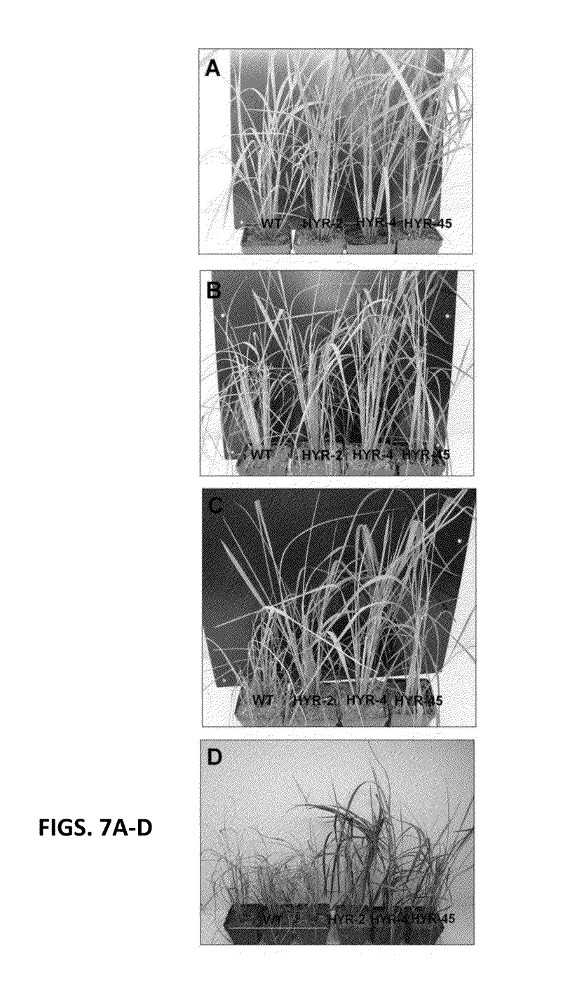

During drought stress treatment, transgenic plants maintained higher relative water content (FIG. 6a) and photosynthesis rates (measured under saturating CO₂ conditions) than WT (FIG. 6b), both of which are key phenotypes related to plant productivity. It is noteworthy to mention that, after eight days of drought stress HYR lines still survived and maintained 65% RWC (FIG. 6a), where as the non transformed WT plants were either dead or nearly dead because of severe loss of water and concomitant damage to the leaves (FIG. 7).

As shown in FIG. 6b, CO₂ gas exchange parameters indicated that HYR lines maintained a significantly higher rate of photosynthetic carbon assimilation compared with WT under both well watered (32%) and drought stress (60%) conditions. This is consistent with gravimetric WUEg estimates (FIG. 3).

### Example 4

### Soluble Carbohydrate Content of HYR Lines

Experiment:

The three selected transformants from previous experiments (HYR-2, HYR-4, and HYR-16) plus the WT were used to examine common photosynthetic biochemical products. The plants were grown as described above but in an environment-controlled growth chamber with the following environmental settings: 14-hr day length, temperature of 28/25° C. day/night, 60% relative humidity, and an irradiance at canopy height of 350 μmol m⁻² s⁻¹ PAR. Plants were grown under these conditions for 8 weeks. At that point, half of the plants in each genotype were drought stressed by withholding water for 3 d. A day before sampling for sugars, pot weights were adjusted to equal weight by adding water as necessary. Soil moisture content (SMC) was determined on the sampling date with a Delta-T theta moisture probe. For sampling of sugars, plants were harvested (all above-ground biomass) and dried at 40° C. for 72 hr. Glucose, fructose, and sucrose were extracted and analyzed according to the procedures of Hendrix (1993) with modifications (Zhang et al., 2006). Sugars were extracted from 20-mg ground samples in 2 ml of 80% ethanol in an 80° C. water bath for 15 min. The crude extract was cooled to room temperature and then centrifuged at 3000 g for 10 min. To 1.5 ml of the supernatant, 20 mg charcoal were added. The extract was centrifuged at 2200 g for 15 min and 20 μl were transferred to a microtiter plate and dried at 50° C. for 1.5 hr. A series of standard solutions of glucose, fructose, and sucrose was co-analyzed with the extracts. After drying, 20 μl deionized-distilled water were added to each well, and the plate was covered for 1 hr. 100 μl of glucose reagent (Sigma, St. Louis, Mo.) were added to each well, and the plate was kept at room temperature for 30 min. Glucose was measured on a microplate reader (SpectroMax plus 386, Molecular Devices Corp. Sunnyvale, Calif.) at 340 nm. 10 μl of 0.25 enzyme unit (EU) phosphoglucose isomerase were added to each well and incubated at room temperature for 30 min, and fructose was measured at 340 nm. 10 μl of 83 EU invertase solution were added and incubated for 30 min before measuring at 340 nm for sucrose.

The experiment was a 2×4 factorial with factors being watering regime and genotype arranged in a completely randomized design (CRD) with four replications. Two-way analysis of variance (ANOVA) to test the effects of drought, genotype, and their interaction was performed using the general linear model (Proc GLM) of Statistical Analysis System (SAS). Differences between means were tested by the Least Significance Difference with a 0.05 threshold (LSD_{0.05}).

Measurement of Glucose, Fructose, and Sucrose Content in HYR Lines.

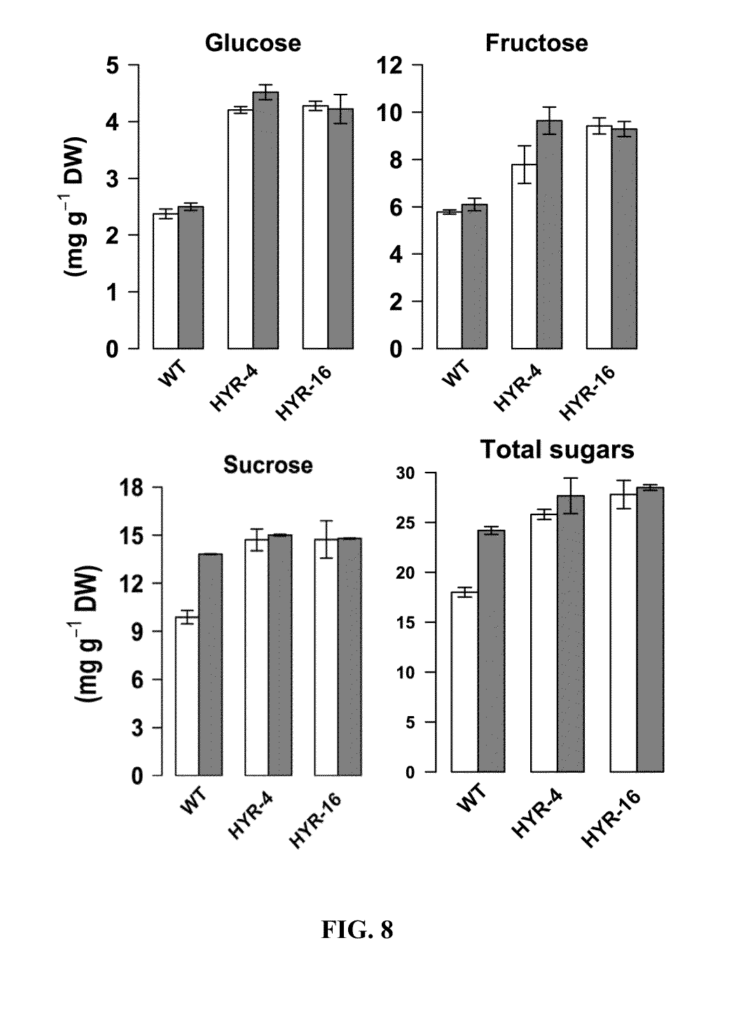

To study more closely the effects of a higher photosynthetic rate on metabolism in two of the HYR lines, an analysis of their soluble carbohydrates was carried out. Plants grown in a controlled environment under well-watered or drought-stressed conditions were harvested, and their dry matter analyzed for glucose, fructose, and sucrose content as described above. In the well-watered controls, the average SMC was 0.41 cm³ cm⁻³, while in the drought stressed plants it was 0.13 cm³ cm⁻³. Drought stress increased glucose, fructose, and sucrose; and there were genotypic differences for these sugars (FIG. 8). However, there were no interactions between genotype and watering regime for any of the sugars (ANOVA data not shown). The two HYR lines produced more free glucose, fructose, sucrose, and total sugars than the WT under well-watered and drought-stressed conditions.

### Example 5

### Grain Yield Under Well-Watered Conditions

Experiment:

The same three transgenic lines and WT plants were grown in the greenhouse in 500-ml pots (one plant per pot) filled with a 1:1 mixture of topsoil and compost (Scott-Sierra Horticultural Product Co, Marysville, Ohio). Pots were placed in a tray filled with water to simulate flooded/paddy conditions. Plants were supplied with a general-purpose (20-20-20) fertilizer (Scott-Sierra Horticultural Product Co, Marysville, Ohio) dissolved in water to provide 50 kg N, P₂O₅, and K₂O ha⁻¹. Fertilizer was applied once a week throughout the growing period. This experiment was carried out between April and September (approximately 14 hr photoperiod), and the greenhouse temperatures were moderated near 28/22° C. day/night.

Plants were grown to maturity (stage R9, with all filled grains having brown hulls) (Counce et al., 2000). The panicle on the main culm was harvested, and spikelets with grains and unfilled spikelets were counted. The grains (caryopses with hulls [palea and lemma] attached) were threshed by hand and dried at 37° C. for 7 d and weighed. The main culm was also harvested and dried at 70° C. for 72 hr and weighed. The yield components assessed were number of spikelets (SP), spikelet fertility (SF) (number of spikelets with filled grains divided by the total number of spikelets), grain yield (GY) (weight of grain), and average single-grain weight (GY divided by grain number). The harvest index (HI) was calculated as the ratio of total grain weight (GY) to total above ground dry weight.

The experiment was a completely randomized design (CRD) with four replicates. Data were subjected to analysis of variance (ANOVA) using the general linear model (Proc GLM) of Statistical Analysis System (SAS). Differences between means were tested by the Least Significance Difference with a 0.05 threshold (LSD_{0.05}).

Grain Yield Measured as Main-Culm Panicle of Well-Watered Plants.

The results to this point showed that HYR lines developed higher biomass, had higher rates of photosynthesis, and contained more soluble sugars. Selected lines (HYR-2, HYR-4, HYR-45) were further analyzed for yield of the main/primary culm and its components when grown under continuously well-watered conditions. The HYR lines had increased main culm biomass, more yield, more grains, and larger single-grain weights relative to the WT (Table 2 shown below). Only HYR-45 had significantly more total spikelets (SP) than the WT. However, spikelet fertility and harvest index were not significantly different in the HYR lines. This means that grain yield increased due to higher single-grain weight and grain number and SP in HYR-45. In summary, the results indicate that the HYR lines produced larger panicles with more and larger grains as well as more total biomass under well-watered conditions.

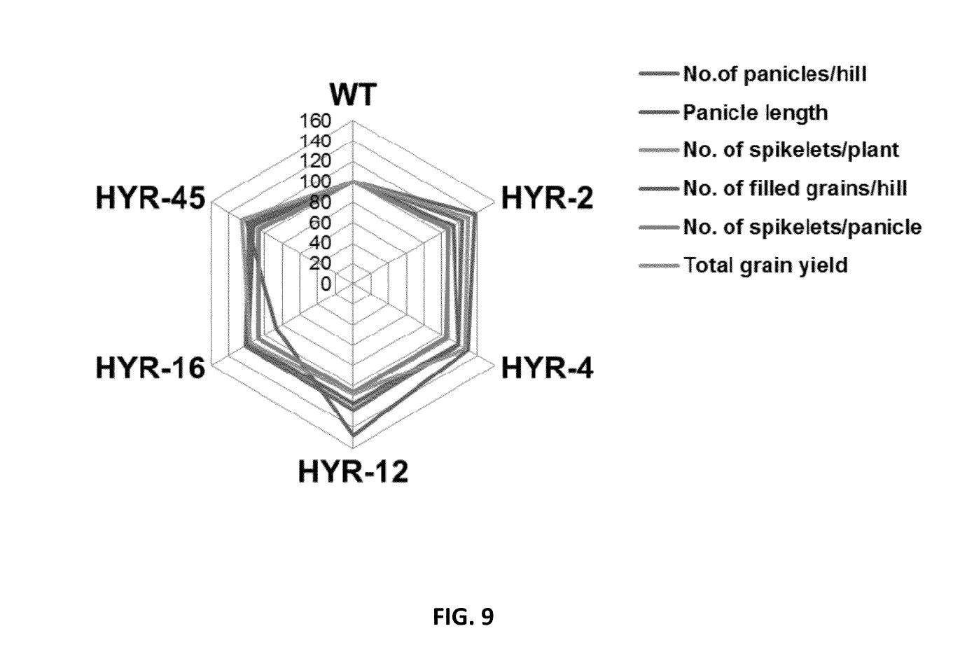

Yield components were evaluated for agronomic traits in the next season for T3 transgenic plants under well-watered conditions. Five independent T3 homozygous lines of the HYR along with WT controls were grown side by side in the green house with the same soil mixture as mentioned above. A completely randomized design was employed with three replicates for each genotype, each replicate consisting of three plants. To evaluate yield components of transgenic plants along with WT, panicles were harvested independently. The filled and unfilled grains were taken apart, independently counted, and weighed. The following agronomic traits were scored: number of panicles/hill, panicle length, number of spikelets/plant, number of filled grains/hill, number of spikelets/panicle and total grain yield. Statistical analysis of the scored yield parameters (FIG. 9) was significantly higher in HYR expressed rice lines than in the WT plants. For example, the number of filled grains are about 20-40% more in HYR lines than in the WT, which resulted in an increase in the total grain weight by 15-30%, depending on the transgenic line (FIG. 9). It was reported that increasing the number of filled grains might be due to the contribution of carbohydrates from current photosynthesis which have been more and efficiently would translocated into the grain and thus increased the grain yield (Xu and Zhou, 2007). The above results clearly indicate that HYR plays a significant role in conferring drought tolerance and improving grain yield in rice. The results from five independent lines were analyzed by two-way ANOVA and compared with those of the WT controls. The ANOVA was used to reject the null hypothesis of equal means of transgenic lines and WT controls. As shown in Table 2 below, plants were grown under well watered conditions until the R9 stage. Means within columns followed by the same letter are not significantly different at P=0.05, with n=4.

TABLE 2
Analysis of shoot biomass and yield components in wild type (WT)
and HYR-transgenic plants

| | Spikelet | Plant | Grain | Grain | Single- | Harvest | Spikelet |
|---|---|---|---|---|---|---|---|
| | Number | Biomass | Number | Yield | Grain | Index | Fertility |
| Genotype | (SP) | (g/plant) | (grain/plant) | (g/plant) | Weight (g) | (HI) | (SF) |
| WT | 381^{b} | 7.32^{c} | 351^{b} | 7.34^{b} | 0.019^{c} | 0.50^{a} | 0.92^{a} |
| HYR-2 | 424^{ab} | 9.33^{ab} | 395^{a} | 8.74^{a} | 0.023^{b} | 0.48^{a} | 0.93^{a} |
| HYR-4 | 430^{ab} | 9.54^{a} | 396^{a} | 8.81^{a} | 0.025^{ab} | 0.48^{a} | 0.91^{a} |
| HYR-45 | 448^{a} | 8.57^{b} | 420^{a} | 9.80^{a} | 0.024^{a} | 0.51^{a} | 0.92^{a} |
| LSD (0.05) | 45.75 | 0.91 | 30.4 | 0.84 | 0.003 | 0.039 | 0.048 |
| P Value | 0.04 | 0.004 | 0.0012 | 0.0042 | 0.012 | 0.60 | 0.48 |

HYR Transgenic Plants have Increased Shoot Biomass and Grain Yield.

All HYR transgenic lines tested accumulated more shoot biomass (FIG. 3), exhibiting larger phenotypes (FIG. 4, 7), and had higher grain yields (Table 2, FIG. 9). To examine possible reasons for the increase in biomass and yield, the HYR lines were further characterized for gas exchange and water-use parameters. The results showed that the transgenic plants had higher net photosynthesis (Pn) compared to the WT. The HYR lines also showed higher water use efficiency (WUEg and WUEi), measured by two independent methods (gravimetric and gas exchange). The higher Pn of HYR lines suggests an explanation for the higher biomass produced. Various studies have shown increased biomass production coinciding with improved Pn in Arabidopsis (Kebeish et al., 2007), mulberry (Morus alba) (Chaitanya et al., 2002), and rice (Karaba et al., 2007). In rice, biomass accumulation before heading affects final yield performance (Chen et al., 2008). The higher grain yields measured in the HYR lines studied under well-watered conditions here were also associated with an increased number of grains and single-grain weights. Taken together, these data suggest a higher Pn during the vegetative stage produced larger HYR plants, which produced more and then larger grains.

The primary stable product of photosynthesis and the phloem-mobile form of sugar is sucrose. High rates of photosynthesis and/or reduced sink sizes can lead to sucrose accumulation and a feedback inhibition of photosynthesis (Vassey and Sharkey, 1989). Sucrose and its immediate metabolic products (glucose and fructose) were therefore examined in selected HYR lines and WT. The analysis shows that the higher-Pn HYR lines accumulated higher levels of sucrose, glucose, fructose, and total soluble sugars than WT. It cannot be determined from the data at hand whether the putative feedback loop (whereby “excess” sucrose inhibits Pn) is less “sensitive” in HYR or if sucrose levels at critical sites (presumably the chloroplasts) are less because of stronger sink activity in the larger, faster growing plants. In either case, it can be reasoned that the higher sugar levels in these plants is probably a direct result of the higher photosynthesis. Higher photosynthesis presumably leads to increased sucrose synthesis and greater export to various sinks. In the soluble sugar study, the plants were in a vegetative stage, when roots are the primary sinks. In a reproductive stage, the larger, more numerous grains would become the primary sinks. In either case, increased sink size might prevent carbohydrate accumulation in the leaves, which could down-regulate photosynthesis (Murchie et al., 1999). Again, though, it cannot be ruled out the possibility that HYR mutants have a reduced sensitivity to sucrose's inhibition of photosynthesis.

### Example 6

### HYR Rice Plants Exhibit Drought Resistance with Improved WUE

The HYR transgenic lines were phenotyped for drought resistance parameters. The progressive drought experiment where watering of plants was stopped, showed a progressive reduction in soil moisture in all genotypes. After around 4 days of drought, the WT are show about 75% RWC and HYR lines maintained 85% RWC. The growth and biomass of the WT is significantly reduced compared to the rice HYR lines (FIG. 7). Compared to the WT, HYR lines had higher photosynthesis (Pn), WUEg, and WUEi under drought stress (FIG. 5), with no significant changes in stomatal conductance (Gs) or transpiration rate (E). Stomatal closure typically leads to decreases in photosynthetic CO₂ assimilation due to restricted diffusion of CO₂ into the leaf and altered CO₂ metabolism. Pelleschi et al. (1997) found that reduced CO₂ diffusion during stomatal closure is mainly responsible for the decline in photosynthesis in C₃ plants subjected to dehydration. In C₃ plants, WUE is determined by, among other factors, stomatal control of the ratio of the instantaneous rates of photosynthesis and transpiration (Farquhar and Sharkey 1982). The results for the HYR plants imply that WUE is not determined by stomatal control of photosynthesis. However, Tezara et al. (1999) reported that, in sunflower (Helianthus annuus) (a C₃ plant) under water stress, the photosynthetic rate is limited more by altered CO₂ metabolism than by reduced diffusion. The results in this study (FIG. 5) suggest that inhibition of photosynthesis under drought stress was not limited by stomatal control, but CO₂ fixation as reported by Tezara et al. (1999). Therefore, CO₂ metabolism in the HYR lines is probably more resistant to dehydration than the WT.

The HYR lines had significantly higher RWC % than the WT under drought stress conditions. Maintenance of plant water status, as expressed by RWC % is an indication of drought resistance (Babu et al., 2003). One of the factors that contributed to high RWC % in the HYR lines could be the accumulation of sugars, especially sucrose, leading to osmotic adjustment. In osmotic adjustment, leaves develop a more negative osmotic potential by accumulating solutes. They can then maintain a higher RWC % during a period of leaf water potential reduction. Solute accumulation and osmotic adjustment have been associated with drought tolerance in ber (Ziziphus mauritiana) (Clifford et al., 1998), pea (Pisum sativum) (Rodriguez-Maribona et al., 1992), tall fescue (Festuca arundinacea) (Elmi and West, 1995), wheat (Morgan, 1995), and sorghum (Sorghum bicolor) (Ludlow et al., 1990).

Soluble carbohydrates (glucose, sucrose, fructose, sorbitol, and mannitol) have been reported to accumulate in plants under drought stress (Abebe et al., 2003; Dancer et al., 1990; Gebre et al., 1998). This is due to a shift in C-partitioning from non-soluble carbohydrates (starch) to soluble carbohydrates, which helps maintain turgor for longer periods during drought (Wang et al., 1995) and participate in stress-protective functions (Abebe et al., 2003).

Overexpression of the HYR transcription factor TF in rice has enabled the plants to be more productive due to efficient mechanisms that enabled the plant to carry out higher levels of Pn and use water more efficiently. The plants are also drought resistant, due to adaptation that enable the plant to continue functioning in the presence of soil water deficit.

### Example 7

### Rice OsHYR Lines have Increased Chlorophyll Content, Robust Root System and Drought Tolerant Phenotype

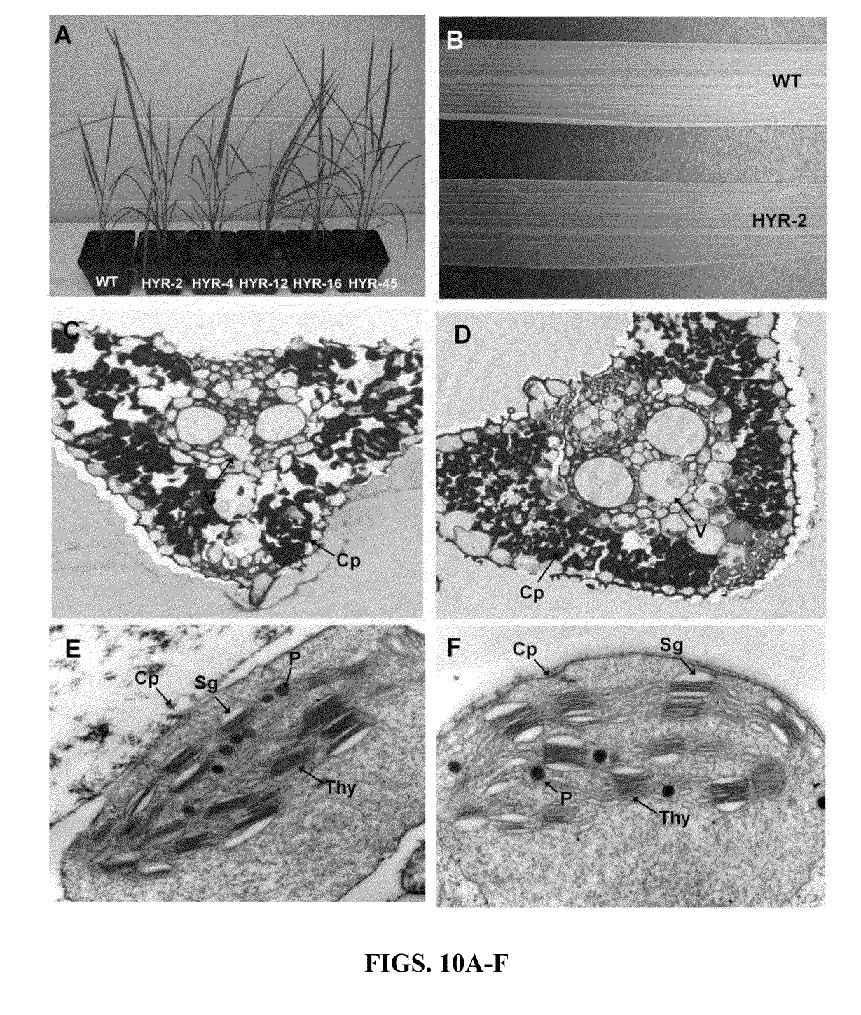

To further investigate the drought stress-tolerance phenotype, chlorophyll content and Fᵥ/Fₘ (where Fv stands for variable fluorescence and Fm stands for maximum fluorescence) values of five rice transgenic HYR lines along with WT control plants were measured. Close inspection of rice lines overexpressing the HYR gene showed a brilliant dark-green leaves (FIG. 10a and b), this dark green margin was sufficient to lead to a measurable increase in total chlorophyll levels in the five independent lines expressing OsHYR. As shown in Table 3 below, the amount of chlorophyll content in HYR lines was significantly increased (about ˜15 %). To confirm this suggestion, under low-magnification confocal microscopy allowed better visualization of mesophyll cells with increased number of chloroplasts in HYR lines than WT (FIG. 10d). The Fv/Fm values represent the maximum photochemical efficiency of PSII in a dark-adapted state. The Fv/Fm levels were about 5% and 35% higher in the HYR lines, than in the WT plants under well-watered and drought stress conditions, respectively (Table 3). Hence, our results indicate that affect of drought-stress in the fluorescence parameter Fv/Fm, which is a measure of accumulated photooxidative damage to PS II, were considerably smaller in the HYR lines than in the WT plants (Table 3). In order to investigate the increased number of chloroplasts and drought tolerance phenotype in HYR leaves, cytological analysis was carried out using transmission electron microscopy (TEM). The TEM analysis revealed that the structure of the chloroplast in HYR lines under drought was not affected (FIG. 10f), where as in WT plants their shapes were changed from oblong to spherical and severely damaged thylakoid membranes (FIG. 10e) have been observed under the same level of drought stress.

As shown in Table 3, chlorophyll content and Fv/Fm values of rice WT and HYR lines are provided. Chlorophyll was extracted from 3-weeks old (250 μmmol m-2 s⁻¹ with a photoperiod of 16-h light/8-h dark cycle) seedlings, and the estimation was done according to Arnon (1949). Chlorophyll fluorescence was measured for both well-watered (WW) and drought (DR) stressed plants using OS1-FL Chlorophyll Fluorometer. Each data point represents the mean±SE (n=6).

TABLE 3
Chlorophyll content and Fv/Fm values of rice WT and HYR lines.

| | Chlorophyll content | Fv/Fm | |
|---|---|---|---|
| Genotype | (mg g⁻¹ FW) | WW | DR |
| WT | 4.03 ± 0.06 | 0.744 ± 0.007 | 0.427 ± 0.009 |
| HYR-2 | 4.83 ± 0.09 | 0.802 ± 0.003 | 0.606 ± 0.004 |
| HYR-4 | 4.67 ± 0.09 | 0.792 ± 0.002 | 0.583 ± 0.010 |
| HYR-12 | 4.60 ± 0.15 | 0.782 ± 0.009 | 0.538 ± 0.023 |
| HYR-16 | 4.56 ± 0.14 | 0.791 ± 0.010 | 0.655 ± 0.017 |
| HYR-45 | 4.60 ± 0.15 | 0.788 ± 0.008 | 0.554 ± 0.005 |

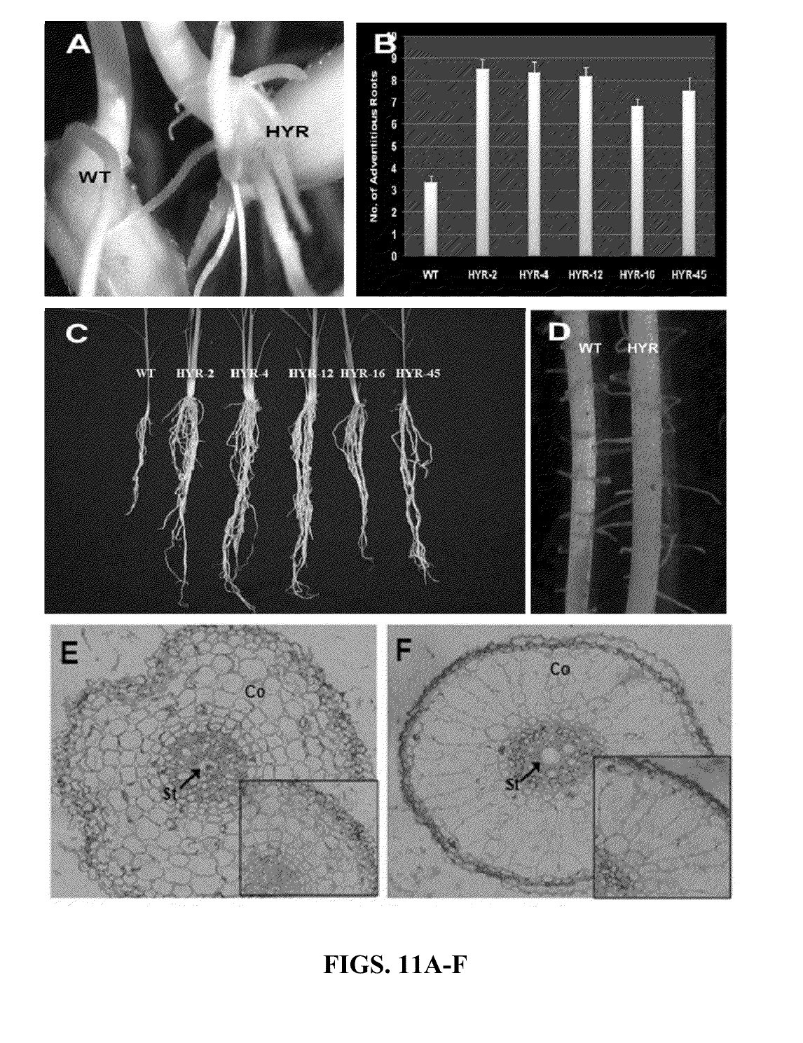

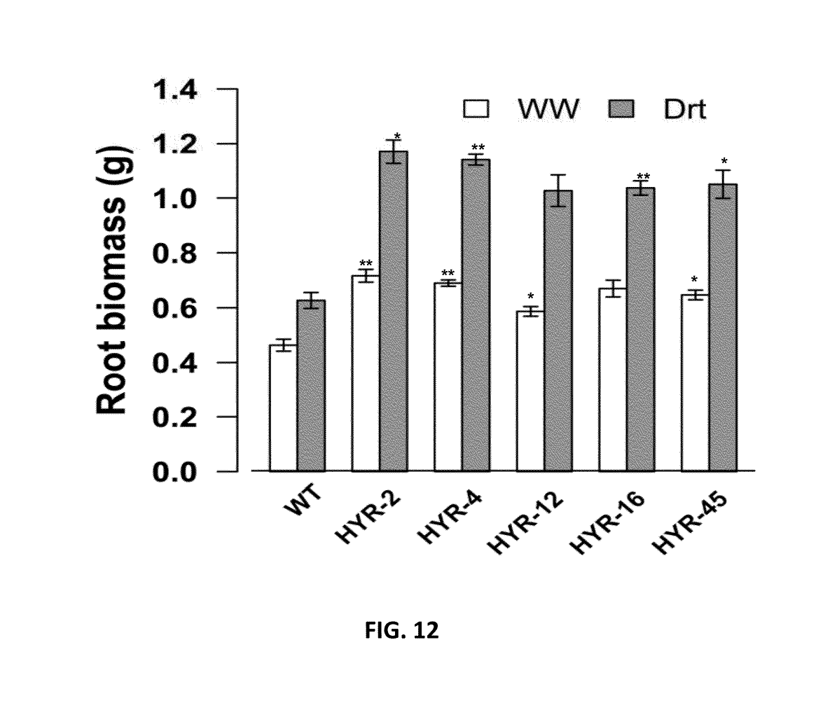

The AP2 transcription factors have been previously found to provide enhanced root strength and increased number of secondary and tertiary roots in transgenic Arabidopsis and rice plants (Karaba et al., 2007; Jeong et al., 2010). Here, it was observed that HYR lines exhibit a robust root system with increased number of adventitious roots (FIG. 11a and b), longer and thicker roots (FIG. 11c and d) than with that of WT plants. In addition, as evidenced in microscopic studies HYR lines showed enlarged stele and larger size of cortical and epidermal cell layers (FIG. 11f) in one-week old grown seedlings on nutrient free medium. It was shown that traits such as maximum root length and adventitious root thickness of rice varieties grown hydroponically can be related to field drought resistance, growth in soil (Price et al., 1997) and grain yield of rice (Jeong et al., 2010). Further, gravimetric analysis of root biomass in HYR lines showed a significant increase of 42% and 72% under well-watered and drought, respectively, compared with that of WT (FIG. 12). It is thought that higher root biomass increases the plant's ability to find less-available water and thus increased drought resistance.

### Example 8

### Expression Analysis of OsHYR in Rice Reveals the Positive Regulation of Photosynthesis and Yield Related Genes

In order to identify genes that are differentially regulated by the over-expression of HYR, expression profiling was performed of 355:OsHYR plants in comparison with WT controls under normal growth conditions at vegetative stage. Gene expression analysis of the rice HYR lines using Affymetrix GeneChips revealed an alteration (both up and down) in several genes; however, the identified genes were further used for gene ontology enrichment analysis for biological processes to determine the pathways/processes associated with OsHYR overexpression. Gene ontology analysis indicated that genes involved in photosynthesis, carbohydrate metabolism and cell cycle were enriched in abundance among the differentially expressed genes. There are about 20 genes belonging to carbohydrate metabolism and photosynthesis that are significantly up-regulated with the P-value≦0.05. Some of these genes are: putative thylakoid lumenal 20 kDa protein (LOC_Os01g59090), photosystem II 11 kD protein (LOC_Os03g21560), magnesium-chelatase subunit chID precursor (LOC_Os03g59640) and chlorophyll a/b binding protein (LOC_Os09g26810). The chlorophyll a/b protein is the major protein component of the light harvesting chlorophyll a/b complex (LHC). In addition, several genes belonging to the glycosyl transferase family are up-regulated. These include the up-regulation of OsGT61-1(LOC_Os02g22380) encoding for xylosyltransferase, which is stress responsive and reported to be expressed more in rice roots than in shoot tissues (Singh et al., 2010).

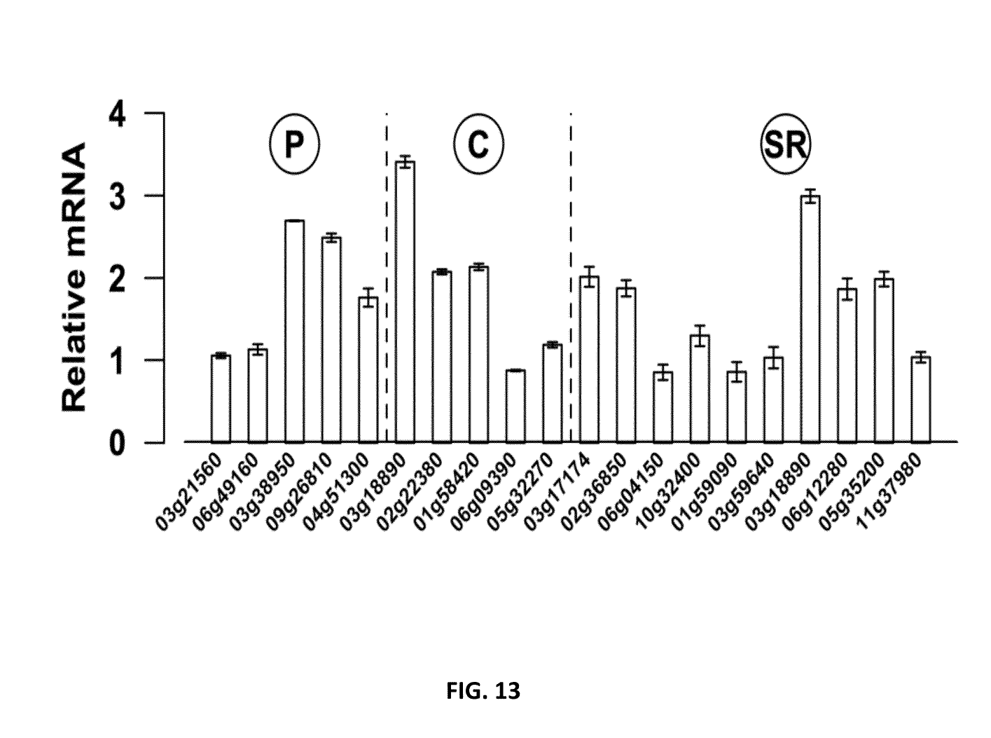

Based on enriched GO terms and important biological processes, candidate genes were selected for quantitative real-time PCR (qRT-PCR) to confirm the microarray analysis (FIG. 13). In order to do qRT-PCR validation, additional biological replicate/samples were used from HYR and WT plants (three replicates in each case) besides the samples used for microarray. The fold-change in expression for selected genes are shown in FIG. 13. The majority of the genes showed high correlation between microarray and qRT-PCR expression values indicated that the expression analysis by both the approaches were in good agreement with each other (FIG. 13). Hence, the results suggest that OsHYR is a key regulator in activating different groups of target genes responsive to drought and yield.

Lack of water is one of the major environmental factors limiting plant productivity. Plant productivity and efficient water use under water-limited conditions are some of the most important traits for drought resistance, especially in crops such as rice (Oryza sativa). Overexpression of the HIGHER YIELD RICE (HYR) gene, an AP2/ERF transcription factor identified from rice drought transcriptome analysis, results in improvement of a combination of agronomic traits. The HYR-overexpressing rice lines have increased shoot biomass, grain yield, and water use efficiency (WUE). The enhanced biomass was correlated with increased net photosynthesis but no greater transpiration, resulting in higher WUE. The increase in net photosynthesis was observed under both well-watered and drought-stressed conditions. The HYR rice lines also accumulated higher levels of soluble sugars (glucose, sucrose, and fructose) and maintained higher relative water content under drought-stress conditions. The results demonstrate the utility of the plant transcription factor HYR for the improvement of plant productivity with or without water-limiting conditions.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. It is intended that the specification and examples be considered as exemplary in nature and that variations that do not depart from the essence of the invention are intended to be within the scope of the invention.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

Additionally, throughout this specification, various publications are referenced. The disclosures of these publications in their entireties are hereby incorporated by reference into this application in order to more fully describe the state of the art to which this pertains and to provide a part of the detailed description. Full citations to some of these references are provided below.
Abebe T, Arron A C, Martin B, Cushman J C (2003) Tolerance of mannitol-accumulating transgenic wheat to water stress and salinity. Plant Physiol 131: 1748-1755.
Arnon D I (1949) Copper enzymes in isolated chloroplasts: polyphenoloxidase in Beta vulgaris. Plant Physiol. 24:1-15.
Babu R C, Zhang J, Blum A, Ho T-H, R W U, Nguyen H T (2004) HVA1, a LEA gene from barley confers dehydration tolerance in transgenic rice (Oryza sativa) via cell membrane protection. Plants Sci 166: 855-862.
Century K, Reuber T L, Ratcliffe O J (2008) Regulating the regulators: the future prospects for transcription-factor-based agricultural biotechnology products. Plant Physiol 147: 20-29.
Chaitanya K V, Masimanani S, Jutur P P, Reddy A R (2002) Variation in photosynthetic rates and biomass productivity among four mulberry cultivars. Photosynthetica 40: 305-308.
Chen S, Zeng F, Pao Z, Zhang Z, Guo Z (2008) Characterization of high-yield performance as affected by genotype and environment in rice. J. Zhejiang Univ Sci B 9: 363-370.
Chuck G, Meely R B, Hake S (1998) The control of maize spikelet meristem fate by APETALA2-like gene indeterminate spikelet1. Genes and Dev 12: 1145-1154.
Clifford S C, Arndt S K, Corlet J E, Joshi S, Sankhla N, Popp M, Jones H G (1998) The role of solute accumulation, osmotic adjustment in drought tolerance in Ziziphus mauritiana (Lamk.). J Exp Bot 49: 967-977.
Counce P A, Keisling C T, Mitchell (2000) A uniform, objective and adaptive system for expressing rice development. Crop Sci 40: 437-443.
Dancer J, David M, Stitt (1990) Water stress leads to a change in partitioning in favor of sucrose in heterotrophic cell suspension culture of Chenopodium rubrum. Plant Cell Environ 13: 957-963.
Elmi A A, West C P (1995) Endophyte infection effects on stomatal conductance, osmotic adjustment and drought recovery of tall fescue. New Phytol 131: 61-67.
Farquhar G D, Sharkey T D (1982) Stomatal conductance and photosynthesis. Annu Rev Plant Physiol Plant Mol Biol 33: 3117-345.
Fujimoto S Y, Ohta M, Usui A, Shinshi H, Ohme-Takagi M (2000) Arabidopsis ethylene-responsive element binding factors act as transcriptional activators or repressors of GCC box-mediated gene expression. Plant Cell 12: 393-404.
Gebre G M, Tschaplisnski T J, Shirshac T L (1998) Water relations of several hardwood species in response to through fall manipulation in upland oak forest during a wet year. Tree Physiol 18: 299-305.
Haake V, Cook D, Riechmann, Pineda O, Thomashaw M F, Zhang J Z (2002) Transcription factor CBF4 is a regulator of drought adaptation in Arabidopsis. Plant Physiol 130: 639-648.
Hendrix D L (1993) Rapid extraction and analysis of nonstructural carbohydrates in plant tissues. Crop Sci 33: 1306-1311.
Hiei Y, Ohta S, Komari T, Kumashiro T (1994) Efficient transformation of Agrobacterium and sequence analysis of the boundaries of the T-DNA. Plant J 6: 271-282.
Horton P (2000) Prospects for crop improvement through genetic manipulation of photosynthesis: morphological and biochemical aspects of light capture. J Exp Bot 51: 475-485.
Jeong J S, Kim Y S, Baek K H, Jung H, Ha S H, Choi Y D, Kim M, Reuzeau C, Kim J K (2010) Root-Specific expression of OsNAC10 improves drought tolerance and grain yield in rice under field drought conditions. Plant physiol. 153:185-197.
Karaba A, Dixit S, Greco R, Aharoni A, Trijatmiko K R, Marsch-Martinez N, Krishnan A, Nataraja K N, Udayakumar M, Pereira A (2007) Improvement of water use efficiency in rice by expression of HARDY, an Arabidopsis drought and salt tolerance gene. Proc Natl Acad Sci USA 104: 15270-15275.
Kebeish R, Niessen M, Thiruveedhi K, Bari R, Hirsh J-H, Rosenkranz R, Stabler N, Schonfeld B, Kreuzaler F, Peterhansel C (2007) Chloroplastic photorespiratory bypass increases photosynthesis and biomass production in Arabidopsis thaliana. Nat Biotechnol 25: 593-9.
Kikuchi S, Satoh K, Nagata T, Kawagashira N, Doi K, Kishimoto N, Yazaki J, Ishikawa M, Yamada H, Ooka H, et al. (2003) Collection, mapping, and annotation of over 28000 cDNA clones from japonica rice. Science 301: 376-379.
Liu Q, Kasuga M, Sakuma Y, Abe H, Miura S, Yamaguchi-Shinozaki K, Shinozaki K (1998) Two transcription factors, DREB1 and DREB2, with an EREBP/AP2 DNA binding domain separate two separate signal transduction pathways in drought- and low-temperature-responsive gene expression, respectively, in Arabidopsis. Plant Cell 10: 1391-1406.
Long S P, Zhu X G, Naidu S L, Ort D R (2006) Can improvement in photosynthesis increase crop yields? Plant Cell Environ 29: 315-330.
Ludlow M M, Santamaria J M, Fukai S (1990) Contribution of osmotic adjustment to grain yield in Sorghum bicolor (L.) Moench under water-limited conditions. II. Water stress after anthesis. Aust J Agr Res 42: 67-78.
Martin B, Thorstensen Y R (1989) Stable carbon isotope composition, water use efficiency, and biomass productivity of Lycopersicon esculentum, Lycopersicon pennellii, and their F1 hybrid. Plant Physiol 88: 213-217.
Morgan J M (1995) Growth and yield of wheat lines with differing osmoregulative capacity at high soil water deficit in seasons of varying evaporative demand. Field Crop Res 40: 143-152.
Murchie E H, Sarrobert C, Contard P, Betsche T, Foyer C H, Galtier N (1999) Overexpression of sucrose-phosphate synthase in tomato plants grown with CO₂ enrichment leads to foliar carbohydrate accumulation relative to untransformed controls. Plant Physiol Bioch 37: 251-260.
Nakano T, Suzuki K, Fujimura T, Shinshi H (2006) Genome-wide analysis of the ERF gene family in Arabidopsis and rice. Plant Physiol 140: 411-432.
Nishimura A, Aichi A, Matsuoka M (2007) A protocol for Agrobacterium-mediated transformation in rice. Nature Protocols 1: 2796-2802.
Oh S-J, Kim Y S, Kwon C-W, Park H K, Jeong J S, Kim J-K (2009) Overexpression of the transcription factor AP37 in rice improves grain yield under drought conditions. Plant Physiol 150: 1368-1379.
Pellegrineschi A, Reynolds M, Pacheco M, Brito R M, Almeraya R, Yamaguchi-Shinozaki K, Hoisington D (2004) Stress-induced expression in wheat of the Arabidopsis thaliana DREB1A gene delays water stress symptoms under greenhouse conditions. Genome 47: 493-500.
Pelleschi S, Rocher J P, Prioul J L (1997) Effect of water restriction on carbohydrate metabolism and photosynthesis in mature maize leaves. Plant Cell Environ 20: 493-503.
Price A H, Tomos A D, Virk D S (1997) Genetic dissection of root growth in rice (Oryza sativa L.). I. A hydrophonic screen. Theor. Appl. Genet. 95:132-142.
Rodriguez-Maribona B, Tenorio J L, Conde J R, Ayerbe L (1992). Correlation between yield and osmotic adjustment of peas (Pisum sativum) under drought stress. Field Crop Res 29: 15-22.
Sakuma Y, Maruyama K, Osakabe Y, Qin F, Seki M, Shinozaki K, Yamaguchi-Shinozakia K (2006) Functional analysis of an Arabidopsis transcription factor, DREB2A, involved in drought-responsive gene expression. Plant Cell 18: 1292-1309.
Selote D S, Khanna-Chopra R (2004) Drought induced spikelet sterility is associated with an inefficient antioxidant defense in rice panicles. Physiol Plant 121: 462-471.
Shinozaki K, Yamaguchi-Shinozaki K, Seki M (2003) Regulatory network of gene expression in the drought and cold stress responses. Curr Opin Plant Biol 6: 410-417.
Singh, A, Singh U, Mittal D, Grover A (2010) Transcript expression and regulatory characteristics of rice glycosyltransferase OsGT61-1 gene. Plant Sci 179: 114-122.
Smart R E, Bingham G E (1974) Rapid estimation of relative water content. Plant Physiol 53: 258-260.
Tezara W, Mitchel V J, Driscoli S D, Lawlor D W (1999) Water deficit inhibits plant photosynthesis by decreasing coupling factor and ATP. Nature 401: 914-917.
Vassey T L, Sharky (1989) Mild water stress of Phaseolus vulgaris plants leads to reduced phosphate synthase activity. Plant Physiol 89: 1066-1070.
Wang Z, Quebedeaux B, Stutte G W (1995) Osmotic adjustment: effect of water stress on carbohydrates in leaves, stems and roots of apple. Australian J Plant Physiol 22: 747-754.
Weigel D (1995) The APETALA2 domain is related to a novel type of DNA binding domain. Plant Cell 7: 388-389.
Xu, ZZ and Zhou G S (2007) Photosynthetic recovery of perennial grass Leymus chinesis after different periods of soil drought. Plant prod. Sci. 10: 277-285.
Zhang X, Ervin E H, LaBranche A J (2006) Metabolic defense responses of seeded bermudagrass during acclimation to freezing stress. Crop Sci 46: 2598-2605.
Zhou J, Xiangfeng W, Jiao Y, Qin Y, Liu X, He K, Chen C, Ma L, Wang J, Xiong L, Zhang Q, Fan L, Deng X W (2007) Global genome expression analysis of rice in response to drought and high salinity stresses in shoot, flag leaf, and panicle. Plant Mol Biol 63: 591-608.
Zhu X G, de Sturler E, Long S P (2007) Optimizing the distribution of resources between enzymes of carbon metabolism can drastically increase photosynthetic rate: a numerical simulation using an evolutionary algorithm. Plant Physiol. 145: 513-526.
Zinselmeier C, Westgate M E, Schussler Jr, R (1995) Low water potential disrupts carbohydrate metabolism in maize (Zea mays L.) ovaries. Plant Physiol 107: 385-391.

1. A transgenic crop plant comprising a chimeric gene that comprises:
a transcription regulatory sequence active in plant cells and
a nucleic acid sequence encoding a HYR protein,
wherein such HYR protein comprises the sequence of SEQ ID NO:1, a sequence with at least 70% similarity to SEQ ID NO:1, a sequence encoding an ortholog protein, a sequence encoding a homologous protein, a functional fragment, and any combination thereof.

2. The transgenic crop plant of claim 1, wherein the transcription regulatory sequence is operably linked to the nucleic acid sequence encoding a HYR protein.

3. The transgenic crop plant according to claim 1, wherein the plant has one or more phenotypes chosen from at least one of enhanced water use efficiency, enhanced photosynthesis, enhanced biomass, enhanced grain yield, and enhanced drought tolerance.

4. The transgenic crop plant according to claim 1, wherein the transcription regulatory sequence is chosen from at least one of a constitutive promoter, an inducible promoter, a tissue-specific promoter, and a developmentally regulated promoter.

5. The transgenic crop plant according to claim 1, wherein the plant is selected from a genus chosen from at least one of Zea, Oryza, Triticum, Solanum, Hordeum, Brassica, Glycine, Phaseolus, Avena, Sorghum, Saccharum, Gossypium, Populus, Quercus, Salix, Miscanthus, and Panicum.

6. A chimeric gene comprising:
a constitutive, inducible, developmental stage-preferred, cell type-preferred, tissue-preferred, or organ-preferred promoter active in plant cells; and
a nucleic acid sequence encoding a HYR protein,
wherein such HYR protein comprises the sequence of SEQ ID NO:1, a sequence with at least 70% similarity to SEQ ID NO:1, the sequence of SEQ ID NO:3, the sequence of SEQ ID NO:4, a sequence encoding an ortholog protein, a functional fragment thereof, and any combination thereof.

7. A vector comprising the chimeric gene of claim 6.

8. A vector according to claim 7, wherein the encoding nucleic acid sequence has the sequence of SEQ ID NO:2, a sequence encoding an ortholog protein, a sequence encoding a homologous protein, a functional fragment, and any combination thereof.

9. A vector according to claim 7, wherein the nucleotide sequence is operably linked with a stress inducible promoter.

10. A method of using a HYR protein according to claim 6 to create a transgenic crop plant that has one or more phenotypes chosen from at least one of enhanced water use efficiency, enhanced photosynthesis, enhanced biomass, enhanced grain yield, and enhanced drought tolerance.

11. The method of claim 10, wherein the transgenic crop plant is selected from a genus chosen from at least one of Zea, Oryza, Triticum, Solanum, Hordeum, Brassica, Glycine, Phaseolus, Avena, Sorghum, Saccharum, Gossypium, Populus, Quercus, Salix, Miscanthus, and Panicum.

12. A method of using a vector according to claim 7 to create a transgenic crop plant that has one or more phenotypes chosen from at least one of enhanced water use efficiency, enhanced photosynthesis, enhanced biomass, enhanced grain yield, and enhanced drought tolerance.

13. The method of claim 12, wherein the transgenic crop plant is selected from a genus chosen from at least one of Zea, Oryza, Triticum, Solanum, Hordeum, Brassica, Glycine, Phaseolus, Avena, Sorghum, Saccharum, Gossypium, Populus, Quercus, Salix, Miscanthus, and Panicum.

14. A method of using a host cell comprising:
a transcription regulatory sequence active in plant cells; and
a nucleic acid sequence encoding a HYR protein,
wherein such HYR protein comprises the sequence of SEQ ID NO:1, a sequence with at least 70% similarity to SEQ ID NO:1, a sequence encoding an ortholog protein, a sequence encoding a homologous protein, a functional fragment, and any combination thereof to create a transgenic crop plant.

15. The method of claim 14, wherein the transcription regulatory sequence is operably linked to the nucleic acid sequence encoding a HYR protein.

16. The method of claim 14, wherein the transgenic crop plant has one or more phenotypes chosen from at least one of enhanced water use efficiency, enhanced photosynthesis, enhanced biomass, enhanced grain yield, and enhanced drought tolerance.

17. The method of claim 14, wherein the transcription regulatory sequence is chosen from at least one of a constitutive promoter, an inducible promoter, a tissue-specific promoter, and a developmentally regulated promoter.

18. The method of claim 14, wherein the transgenic crop plant is chosen from at least one of Zea, Oryza, Triticum, Solanum, Hordeum, Brassica, Glycine, Phaseolus, Avena, Sorghum, Saccharum, Gossypium, Populus, Quercus, Salix, Miscanthus, and Panicum.

19. A seed or a fruit of a plant according to claim 1.

20-21. (canceled)
